SEQUENCE LISTING (72 sequences)

SEQ ID NO: 1 - DNA, 208 bp, Homo sapiens
gttccaaaaacagtggataattttgtggccttagctacaggagagaaaggatttggctacaaaaacagcaaattccatcgtgtaatcaaggacttcatgatccagggcggagacttcaccaggggagatggcacaggaggaaagagcatctacggtgagcgcttccccgatgagaacttcaaactgaagcactacgggcctggctggg

SEQ ID NO: 2 - DNA, 200 bp, Photinus pyralis
tgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagg

SEQ ID NO: 3 - DNA, 19 bp, Artificial sequence
  siRNA sequence with homology to human cyclophilin gene
caaaaacagtggataattt

SEQ ID NO: 4 - DNA, 19 bp, Artificial sequence
  siRNA sequence with homology to human cyclophilin gene
ggccttagctacaggagag

SEQ ID NO: 5 - DNA, 19 bp, Artificial sequence
  siRNA sequence with homology to human cyclophilin gene
ctacaggagagaaaggatt

SEQ ID NO: 6 - DNA, 19 bp, Artificial sequence
  siRNA sequence with homology to human cyclophilin gene
gagagaaaggatttggcta

SEQ ID NO: 7 - DNA, 19 bp, Artificial sequence
  siRNA sequence with homology to human cyclophilin gene
gagaaaggatttggctaca

SEQ ID NO: 8 - DNA, 19 bp, Artificial sequence
  siRNA sequence with homology to human cyclophilin gene
gaaaggatttggctacaaa

SEQ ID NO: 9 - DNA, 19 bp, Artificial sequence
  siRNA sequence with homology to human cyclophilin gene
acagcaaattccatcgtgt

SEQ ID NO: 10 - DNA, 19 bp, Artificial sequence
  siRNA sequence with homology to human cyclophilin gene
caaattccatcgtgtaatc

SEQ ID NO: 11 - DNA, 19 bp, Artificial sequence
  siRNA sequence with homology to human cyclophilin gene
tcatgatccagggcggaga

SEQ ID NO: 12 - DNA, 19 bp, Artificial sequence
  siRNA sequence with homology to human cyclophilin gene
gatccagggcggagacttc

SEQ ID NO: 13 - DNA, 19 bp, Artificial sequence
  siRNA sequence with homology to human cyclophilin gene
gcacaggaggaaagagcat

SEQ ID NO: 14 - DNA, 19 bp, Artificial sequence
  siRNA sequence with homology to human cyclophilin gene
ggaaagagcatctacggtg

SEQ ID NO: 15 - DNA, 19 bp, Artificial sequence
  siRNA sequence with homology to human cyclophilin gene
gcgcttccccgatgagaac

SEQ ID NO: 16 - DNA, 19 bp, Artificial sequence
  siRNA sequence with homology to human cyclophilin gene
aaaacagtggataattttg

SEQ ID NO: 17 - DNA, 19 bp, Artificial sequence
  siRNA sequence with homology to human cyclophilin gene
aacagtggataattttgtg

SEQ ID NO: 18 - DNA, 19 bp, Artificial sequence
  siRNA sequence with homology to human cyclophilin gene
cagtggataattttgtggc

SEQ ID NO: 19 - DNA, 19 bp, Artificial sequence
  siRNA sequence with homology to human cyclophilin gene
ataattttgtggccttagc

SEQ ID NO: 20 - DNA, 19 bp, Artificial sequence
  siRNA sequence with homology to human cyclophilin gene
ttgtggccttagctacagg

SEQ ID NO: 21 - DNA, 19 bp, Artificial sequence
  siRNA sequence with homology to human cyclophilin gene
ttagctacaggagagaaag

SEQ ID NO: 22 - DNA, 19 bp, Artificial sequence
  siRNA sequence with homology to human cyclophilin gene
atttggctacaaaaacagc

SEQ ID NO: 23 - DNA, 19 bp, Artificial sequence
  siRNA sequence with homology to human cyclophilin gene
aaaaacagcaaattccatc

SEQ ID NO: 24 - DNA, 19 bp, Artificial sequence
  siRNA sequence with homology to human cyclophilin gene
aaacagcaaattccatcgt

SEQ ID NO: 25 - DNA, 19 bp, Artificial sequence
  siRNA sequence with homology to human cyclophilin gene
ttccatcgtgtaatcaagg

SEQ ID NO: 26 - DNA, 19 bp, Artificial sequence
  siRNA sequence with homology to human cyclophilin gene
atgatccagggcggagact

SEQ ID NO: 27 - DNA, 19 bp, Artificial sequence
  siRNA sequence with homology to human cyclophilin gene
tccagggcggagacttcac

SEQ ID NO: 28 - DNA, 19 bp, Artificial sequence
  siRNA sequence with homology to human cyclophilin gene
cagggcggagacttcacca

SEQ ID NO: 29 - DNA, 19 bp, Artificial sequence
  siRNA sequence with homology to human cyclophilin gene
acttcaccaggggagatgg

SEQ ID NO: 30 - DNA, 19 bp, Artificial sequence
  siRNA sequence with homology to human cyclophilin gene
ttcaccaggggagatggca

SEQ ID NO: 31 - DNA, 19 bp, Artificial sequence
  siRNA sequence with homology to human cyclophilin gene
ccaggggagatggcacagg

SEQ ID NO: 32 - DNA, 19 bp, Artificial sequence
  siRNA sequence with homology to human cyclophilin gene
tctacggtgagcgcttccc

SEQ ID NO: 33 - DNA, 19 bp, Artificial sequence
  siRNA sequence with homology to human cyclophilin gene
tacggtgagcgcttccccg

SEQ ID NO: 34 - DNA, 19 bp, Artificial sequence
  siRNA sequence with homology to human cyclophilin gene
tgagaacttcaaactgaag

SEQ ID NO: 35 - DNA, 19 bp, Artificial sequence
  siRNA sequence with homology to human cyclophilin gene
agaacttcaaactgaagca

SEQ ID NO: 36 - DNA, 19 bp, Artificial sequence
  siRNA sequence with homology to human cyclophilin gene
tcaaactgaagcactacgg

SEQ ID NO: 37 - RNA, 19 nt, Artificial sequence
  siRNA sequence with homology to human survivin gene
gcaaugucuuaggaaagga

SEQ ID NO: 38 - RNA, 19 nt, Artificial sequence
  siRNA sequence with homology to human survivin gene
agaauagcacaaacuacaa

SEQ ID NO: 39 - RNA, 19 nt, Artificial sequence
  siRNA sequence with homology to human survivin gene
gagacagaauagagugaua

SEQ ID NO: 40 - RNA, 19 nt, Artificial sequence
  siRNA sequence with homology to human survivin gene
gcgucuggcagauacuccu

SEQ ID NO: 41 - RNA, 19 nt, Artificial sequence
  siRNA sequence with homology to human survivin gene
ugcgcuuuccuuucuguca

SEQ ID NO: 42 - RNA, 19 nt, Artificial sequence
  siRNA sequence with homology to human survivin gene
gaagcaguuugaagaauua

SEQ ID NO: 43 - RNA, 19 nt, Artificial sequence
  siRNA sequence with homology to human survivin gene
ugucuuaggaaaggagauc

SEQ ID NO: 44 - RNA, 19 nt, Artificial sequence
  siRNA sequence with homology to human survivin gene
ggcagugucccuuuugcua

SEQ ID NO: 45 - RNA, 19 nt, Artificial sequence
  siRNA sequence with homology to human survivin gene
aauucacagaauagcacaa

SEQ ID NO: 46 - RNA, 19 nt, Artificial sequence
  siRNA sequence with homology to human survivin gene
aagcacaaagccauucuaa

SEQ ID NO: 47 - RNA, 19 nt, Artificial sequence
  siRNA sequence with homology to human survivin gene
ggcaguggccuaaauccuu

SEQ ID NO: 48 - RNA, 19 nt, Artificial sequence
  siRNA sequence with homology to human survivin gene
ggcugaagucuggcguaag

SEQ ID NO: 49 - RNA, 19 nt, Artificial sequence
  siRNA sequence with homology to human survivin gene
gcugaagucuggcguaaga

SEQ ID NO: 50 - RNA, 19 nt, Artificial sequence
  siRNA sequence with homology to human survivin gene
cggcuguuccugagaaaua

SEQ ID NO: 51 - RNA, 19 nt, Artificial sequence
  siRNA sequence with homology to human survivin gene
aaggaccaccgcaucucua

SEQ ID NO: 52 - RNA, 19 nt, Artificial sequence
  siRNA sequence with homology to human survivin gene
ggaccaccgcaucucuaca

SEQ ID NO: 53 - RNA, 19 nt, Artificial sequence
  siRNA sequence with homology to human survivin gene
ccgguugcgcuuuccuuuc

SEQ ID NO: 54 - RNA, 19 nt, Artificial sequence
  siRNA sequence with homology to human survivin gene
gcugcuucucucucucucu

SEQ ID NO: 55 - RNA, 19 nt, Artificial sequence
  siRNA sequence with homology to human survivin gene
gugaugagagaauggagac

SEQ ID NO: 56 - RNA, 19 nt, Artificial sequence
  siRNA sequence with homology to human survivin gene
ggagacagaauagagugau

SEQ ID NO: 57 - RNA, 19 nt, Artificial sequence
  siRNA sequence with homology to human survivin gene
ccuucacaucugucacguu

SEQ ID NO: 58 - RNA, 19 nt, Artificial sequence
  siRNA sequence with homology to human survivin gene
gauuguuacagcuucgcug

SEQ ID NO: 59 - RNA, 19 nt, Artificial sequence
  siRNA sequence with homology to human survivin gene
ucaaggaccaccgcaucuc

SEQ ID NO: 60 - RNA, 19 nt, Artificial sequence
  siRNA sequence with homology to human survivin gene
gaccaccgcaucucuacau

SEQ ID NO: 61 - RNA, 19 nt, Artificial sequence
  siRNA sequence with homology to human survivin gene
aagcauucguccgguugcg

SEQ ID NO: 62 - RNA, 19 nt, Artificial sequence
  siRNA sequence with homology to human survivin gene
uucguccgguugcgcuuuc

SEQ ID NO: 63 - RNA, 19 nt, Artificial sequence
  siRNA sequence with homology to human survivin gene
acugcgaagaaagugcgcc

SEQ ID NO: 64 - RNA, 19 nt, Artificial sequence
  siRNA sequence with homology to human survivin gene
gaaggcagugucccuuuug

SEQ ID NO: 65 - RNA, 19 nt, Artificial sequence
  siRNA sequence with homology to human survivin gene
gacagcuuuguucgcgugg

SEQ ID NO: 66 - RNA, 19 nt, Artificial sequence
  siRNA sequence with homology to human survivin gene
ugugucuggaccucauguu

SEQ ID NO: 67 - RNA, 19 nt, Artificial sequence
  siRNA sequence with homology to human survivin gene
acuaagcacaaagccauuc

SEQ ID NO: 68 - RNA, 19 nt, Artificial sequence
  siRNA sequence with homology to human survivin gene
agccauucuaagucauugg

SEQ ID NO: 69 - RNA, 19 nt, Artificial sequence
  siRNA sequence with homology to human survivin gene
gccauucuaagucauuggg

SEQ ID NO: 70 - RNA, 19 nt, Artificial sequence
  siRNA sequence with homology to human survivin gene
cacugcugugugauuagac

SEQ ID NO: 71 - RNA, 19 nt, Artificial sequence
  siRNA sequence with homology to human survivin gene
uuaaaugacuuggcucgau

SEQ ID NO: 72 - RNA, 19 nt, Artificial sequence
  siRNA sequence with homology to human survivin gene
ccaaccuucacaucuguca

(19) United States
(12) Patent Application Publication
(10) Pub. No.: US 2009/0155904 A1
(43) Pub. Date: Jun. 18, 2009

(54) METHOD OF INHIBITING EXPRESSION OF TARGET MRNA USING SIRNA CONSISTING OF NUCLEOTIDE SEQUENCE COMPLEMENTARY TO SAID TARGET MRNA

(75) Inventors: Young-Chul Choi, Daejeon (KR); Han Oh Park, Deajeon (KR); Sorim Choung, Daejeon (KR); Young Joo Kim, Daejeon (KR); Sang Soo Kim, Deajeon (KR); Seong-min Park, Daejeon (KR); Sang-Cheol Kim, Seoul (KR); Gyuman Yoon, Daejeon (KR); Kyoung Oak Choi, Seoul (KR); Hyo Jin Kang, Daejeon (KR)

Correspondence Address: ROTHWELL, FIGG, ERNST & MANBECK, P.C., 1425 K STREET, N.W., SUITE 800, WASHINGTON, DC, 20005, US

(73) Assignee: Bioneer Corporation, Daejeon (KR)

(21) Appl. No.: 11/721,303

(22) PCT Filed: Dec. 8, 2005
(86) PCT No.: PCT/KR2005/004207

§ 371 Date: Jun. 8, 2007

(30) Foreign Application Priority Data
Dec. 8, 2004 (KR) 10-2004-0103283

(51) Int. Cl. C12N 5/02

(52) U.S. Cl. 435/375

(57) ABSTRACT

A inhibition method of target mRNA expression includes: (a) obtaining binding energy of a double combination section on a dsRNA sequence of all combination comprising complementary nucleotides to a random target mRNA; (b) dividing the binding energy into four sections on the dsRNA sequence of each combination to obtain a difference of the mean binding energy between each section and convert into a score of a relative combination energy pattern; (c) selecting siRNA whose inhibition efficiency to target mRNA is expected to be high by applying the converted score to the dsRNA sequence with other factors that affect the efficiency of siRNA; and (d) inhibiting target mRNA expression using the selected siRNA. As a result, a researcher or an experimenter can analyze patterns of a relative binding energy on base sequences of unknown siRNA without actual experiments to determine whether the siRNA is effective or ineffective rapidly, thereby design and production efficiency of siRNA can be maximized and target mRNA can be effectively inhibited with efficient siRNA to the target mRNA.

### TECHNICAL FIELD

The present invention generally relates to a inhibition method of target mRNA expression using small interfering RNA (hereinafter, referred to as “siRNA”), and more specifically, to a inhibition method of target mRNA expression using siRNA comprising the steps of selecting complementary siRNA predicted to show the maximal target inhibition efficiency by analyzing a relative binding energy pattern between adjacent and nonadjacent portions of nucleotide sequence of candidate siRNAs and inhibiting target mRNA expression by treating said selected siRNA.

### BACKGROUND OF THE INVENTION

RNA interference (hereinafter, referred to as “RNAi”) refers to a phenomenon of decomposing target mRNA in a cytoplasm by double-stranded RNA (hereinafter, referred to as “dsRNA”) having complementary nucleotide sequence of the target mRNA. After first discovered in C. elegans by Fire and Mello in 1998, RNAi phenomenon has been reported to occur in Drosophila, Trypanosoma (a kind of Mastigophora) and vertebrates (Tabara H, Grishok A, Mello C C, Science, 282(5388), 430-1, 1998). In case of human, it was difficult to obtain RNAi effect due to the induction of antiviral interferon pathway upon dsRNA introduction. In 2001, Elbashir and Tuschl et al., reported that the introduction of small dsRNA of 21 nucleotides length into human cells did not cause the interferon pathway but specifically decomposed complementary target mRNA (Elbashir, S. M., Harborth, J., Lendeckel, W., Yalcin, A., Weber, K., Tuschl, T., Nature, 411, 494-498, 2001; Elbashir, S. M., Lendeckel, W., Tuschl, T., Genes & Dev., 15, 188-200, 2001; Elbashir, S. M., Martinez, J., Patkaniowska, A., Lendeckel, W., Tuschl, T., EMBO J., 20, 6877-6888, 2001). Thereafter, dsRNA of 21 nt length has been spotlighted as a tool of new functional genomics and named as small interfering RNA (hereinafter, referred to as “siRNA”). The small interfering RNA (siRNA and microRNA) was granted to the No. 1 of Breakthrough of the Year of the Science Journal in 2002 year (Jennifer Couzin, BREAKTHROUGH OF THE YEAR: Small RNAs Make Big Splash, Jennifer Couzin, Science 20 Dec. 2002: 2296-2297).

siRNA has some advantages as a tool of therapeutics and functional genomics over conventional antisense RNA. First, while antisense RNA requires to synthesize many kinds of antisense RNAs and to perform experiments with a lot of times and costs so as to obtain an effective target sequences, the efficiency of siRNA can be predicted using some algorithms so that more efficient siRNA may be selected through the smaller number of experiments. Second, siRNA has been known to inhibit the expression of genes effectively at a lower concentration than antisense RNA. It means that a smaller amount of siRNA can be used for study and higher therapeutic effect can be expected. Third, inhibition of gene expression by RNAi is a natural mechanism in a body and its action is very specific.

Generally, RNAi experiment includes siRNA design (target site selection), cell culture experiment (cell culture assay, target mRNA degradation rate, the most effective siRNA selection), animal experiment (stability, modification, delivery, pharmacokinetics, toxicology) and clinical test. Of these experiments, the most important step is selecting effective siRNA sequence(s) and delivering selected siRNA into a target tissue (drug delivery). The selection of siRNA sequence having high efficiency is important because different siRNAs show different efficiency and only a siRNA having high efficiency results in an accurate experimental result and can be used for therapy. The efficient nucleotide sequence can be selected by a computer-aided scoring method and an experimental method. The experimental method is directed to select nucleotide sequences that combine well with target mRNA synthesized by in vitro transcription. However, the mRNA structure obtained from in vitro transcription may be different from that of the mRNA in a cell, and various proteins may be bonded to the mRNA in a cell so that a result obtained from the experiment using mRNA obtained by in vitro transcription may not reflect an actual result. Therefore, developing an algorithm for searching an effective siRNA sequence is important and this can be done by considering various elements that influence the effectiveness of siRNA sequence.

Generally, conventional siRNA design has been performed according to the Tuschl rule which considers 3′overhang type, GC ratio, repetition of specific nucleotide, SNP (single nucleotide polymorphism) in a sequence, secondary structure of RNA, homology with un-targeted mRNA sequence (S. M. Elbashir, J. Harborth, W. Lendeckel, A. Yalcin, Klaus Weber, T. Tuschl, Nature, 411, 494-498, 2001a; S. M. Elbashir, W. Lendeckel, T. Tuschl, Genes & Dev., 15, 188-200, 2001b; S. M. Elbashir, J. Martinez, A. Patkaniowska, W. Lendeckel, T. Tuschl, EMBO J., 20, 6877-6888, 2001c). However, binding energy status in a double-stranded part of siRNA has recently been considered in the siRNA design (Khvorova, A., Reynolds, A., Jayasena, S. D., Cell, 115(4), 505, 2003; Reynolds, A., Leake, D., Boese, Q., Scaringe, S., Marshall, W. S., Khvorova, A., Nat. Biotechnol., 22(3), 326-330, 2004). For example, considering that the efficiency of siRNA could be affected critically by which strand of double-stranded siRNA is bonded with RISC(RNAi-induced silencing complex), siRNA efficiency could be predicted by calculating the energy differences between 5′-end and 3′-end of candidate siRNA (Schwarz D S, Hutvagner G, Du T, Xu Z, Aronin N, Zamore P D., Cell, 115(2), 199-208, 2003, see FIG. 1).

The present inventors have studied the relationship between the efficiency of siRNA and the binding energy status of the entire double-stranded parts of siRNA more accurately and precisely using statistical method. Until now, said relationship has only been reported for the partial parts of the siRNA. As a result, we have found that the inhibition efficiency of candidate siRNA on target mRNA can be predicted through pattern analysis of the relative binding energy of the candidate siRNA, and that the expression of target mRNA can be effectively inhibited using the selected siRNA.

### DETAILED DESCRIPTION OF THE PREFERRED EMBODIMENTS

The present invention is directed to provide a method of effectively inhibiting the expression of target mRNA using siRNA selected by analyzing a relative binding energy pattern of candidate siRNA without any experiment.

According to an embodiment of the present invention, an inhibition method of target mRNA expression using siRNA comprises:

(1) obtaining all combinations of dsRNA sequences each of which consists of n numbers of nucleotides complementary to a predetermined target mRNA (n is an integer);

(2) obtaining E_{A}, E_{B}, E_{C} and E_{D} with respect to each dsRNA, which are mean binding energy values of 1^{st}-2^{nd} (A), 3^{rd}-7^{th} (B), 8^{th}-15^{th} (C) and 16^{th}-18^{th} (D) in the base sequence of the dsRNA,

(3) allotting Y_{(A-B)}, Y_{(B-C)}, Y_{(C-D)} and Y_{(A-D)} to each section of (A) through (D) according to the following equation for each of the combination of dsRNA sequence,

for the section (A-B),

$$i ) if {E}_{f ( A - B )} - 1.96 \sqrt{\frac{{S}_{f ( A - B )}}{{N}_{f}}} < {X}_{( A - B )} < {E}_{f ( A - B )} + 1.96 \sqrt{\frac{{S}_{f ( A - B )}}{{N}_{f}}} , then {Y}_{( A - B )} = 10 point , \\ ii ) if {E}_{n ( A - B )} - 1.96 \sqrt{\frac{{S}_{n ( A - B )}}{{N}_{n}}} < {X}_{( A - B )} < {E}_{n ( A - B )} + 1.96 \sqrt{\frac{{S}_{n ( A - B )}}{{N}_{n}}} , then {Y}_{( A - B )} = 0 point ,$$

iii) if X_{(A-B)} does not belong to said ranges, then Y_{(A-B)}=5 point, in the same way, allotting Y_{(B-C)}, Y_{(C-D)} and Y_{(A-D)} for the sections (B-C), (C-D) and (A-D),
wherein E_{i(A-B)} is a mean value of the difference of mean energy value for each section (A-B),
S_{i(A-B)} is a distribution value of the E_{i(A-B)},
Nᵢ is the number of experimental data of siRNA,
X_{(A-B)} is a value corresponding to a difference between mean binding energy E_{A} of the section (A) and mean binding energy E_{B} of the section (B), and the same goes for Y_{(B-C)}, Y_{(C-D)} and Y_{(A-D)};

(4) allotting a relative binding energy Y value by the following Equation 4 with respect to each dsRNA:

$$Y = \frac{\begin{matrix}{W}_{( A - B )} {Y}_{( A - B )} + {W}_{( B - C )} {Y}_{( B - C )} + \\ {W}_{( C - D )} {Y}_{( C - D )} + {W}_{( A - D )} {Y}_{( A - D )}\end{matrix}}{10 ( {W}_{( A - B )} + {W}_{( B - C )} + {W}_{( C - D )} + {W}_{( A - D )} )} \times 100 \qquad [ Equation 4 ]$$

wherein W_{(A-B)} is weight for the section (A-B);

(5) allotting Z value by the following Equation 5 with respect to each dsRNA:

$$Z = 100 \times \frac{{\sum }_{i} {W}_{i} \frac{{Z}_{i}}{{M}_{i}}}{{\sum }_{i} {W}_{i}} \qquad [ Equation 5 ]$$

wherein i is an integer representing a factor affecting siRNA's inhibition efficiency on the target mRNA, at least one of which is the relative binding energy of the siRNA,

Zᵢ is a point given to each factor, provided that Z₁=Y, representing a relative binding energy of step (4),

Mᵢ is a predetermined maximum value allotted to each factor, and

Wᵢ is a predetermined weight allotted to each factor based on W₁;

(6) arranging Z values obtained from the step (5) in a descending order with respect to each dsRNA to select predetermined top % of dsRNAs; and

(7) applying the selected dsRNAs to inhibit the target mRNA expression.

The siRNA is dsRNA comprising 21˜23, preferably 21 nucleotides and has the structure of double stranded central region consisting of 19 nucleotides and an overhanging 1˜3, preferably 2 nucleotides at both 3′ ends of the double stranded central region (see FIG. 3).

In order to optimize the design of siRNA for target mRNA by analyzing relative binding energy pattern of candidate siRNAs which inhibits the expression of the target mRNA, the present inventors have scored and systematized the siRNAs depending on the relative binding energy pattern of the double-stranded region of the siRNAs.

In order to find out the inhibition efficiency of a certain siRNA to target mRNA, the present inventors have examined the correlation between the binding energy status and the inhibition efficiency of the siRNA. The present inventors have focused not on an absolute binding energy value of specific regions of the double-stranded siRNA but on a variation of the relative binding energy between adjacent and nonadjacent parts of the siRNA (see FIG. 2).

According to one embodiment of the present invention, gene expression inhibition data using siRNA are collected from two papers. The one is from Khvorova's paper (Khvorova A, Reynolds A, Jayasena S D, Cell, 115(4), 505, 2003) and the other is from Amarzguioui's paper (Amarzguioui M, Prydz H, Biochem. Biophys. Res. Commun., 316(4), 1050-8, 2004). Khvorova's paper discloses a nucleotide sequence represented by the SEQ. ID. NO:1 corresponding to 193-390 nucleotide sequence of human cyclophilin gene (hCyPB), a nucleotide sequence represented by the SEQ. ID. NO:2 corresponding to 1434-1631 nucleotide sequence of firefly luciferase gene (GL3), and siRNAs for inhibiting the genes. Amarzguioui's paper discloses siRNAs for inhibiting various genes (AA). From the collected data, the base sequence of siRNA used in data analysis and the inhibition effect of gene expression of the siRNA are obtained. Table 1 shows a part of experimental data obtained from Khvorova's paper. INN-HB nearest neighbor model renders information of the base sequences into data on the binding energy (Xia T, SantaLucia J Jr, Burkard M E, Kierzek R, Schroeder S J, Jiao X, Cox C, Turner D H, Biochemistry, 37(42), 14719-35, 1998, see FIGS. 3 and 4).

TABLE 1

| | | | | |
|---|---|---|---|---|
| | | | SEQ ID | Knock- |
| Gene | Position | Sequence* | NO. | down % |

| | | | | |
|---|---|---|---|---|
| hCyPB | 5(+192) | CAAAAACAGTGGATAATTT | 3 | >90 |
| M60857 | 27(+192) | GGCCTTAGCTACAGGAGAG | 4 | >90 |
| | 35(+192) | CTACAGGAGAGAAAGGATT | 5 | >90 |
| | 41(+192) | GAGAGAAAGGATTTGGCTA | 6 | >90 |
| | 43(+192) | GAGAAAGGATTTGGCTACA | 7 | >90 |
| | 45(+192) | GAAAGGATTTGGCTACAAA | 8 | >90 |
| | 65(+192) | ACAGCAAATTCCATCGTGT | 9 | >90 |
| | 69(+192) | CAAATTCCATCGTGTAATC | 10 | >90 |
| | 95(+192) | TCATGATCCAGGGCGGAGA | 11 | >90 |
| | 99(+192) | GATCCAGGGCGGAGACTTC | 12 | >90 |
| | 131(+192) | GCACAGGAGGAAAGAGCAT | 13 | >90 |
| | 139(+192) | GGAAAGAGCATCTACGGTG | 14 | >90 |
| | 159(+192) | GCGCTTCCCCGATGAGAAC | 15 | >90 |
| | 7(+192) | AAAACAGTGGATAATTTTG | 16 | <50 |
| | 9(+192) | AACAGTGGATAATTTTGTG | 17 | <50 |
| | 11(+192) | CAGTGGATAATTTTGTGGC | 18 | <50 |
| | 17(+192) | ATAATTTTGTGGCCTTAGC | 19 | <50 |
| | 23(+192) | TTGTGGCCTTAGCTACAGG | 20 | <50 |
| | 31(+192) | TTAGCTACAGGAGAGAAAG | 21 | <50 |
| | 51(+192) | ATTTGGCTACAAAAACAGC | 22 | <50 |
| | 61(+192) | AAAAACAGCAAATTCCATC | 23 | <50 |
| | 63(+192) | AAACAGCAAATTCCATCGT | 24 | <50 |
| | 73(+192) | TTCCATCGTGTAATCAAGG | 25 | <50 |
| | 97(+192) | ATGATCCAGGGCGGAGACT | 26 | <50 |
| | 101(+192) | TCCAGGGCGGAGACTTCAC | 27 | <50 |
| | 103(+192) | CAGGGCGGAGACTTCACCA | 28 | <50 |
| | 113(+192) | ACTTCACCAGGGGAGATGG | 29 | <50 |
| | 115(+192) | TTCACCAGGGGAGATGGCA | 30 | <50 |
| | 119(+192) | CCAGGGGAGATGGCACAGG | 31 | <50 |
| | 149(+192) | TCTACGGTGAGCGCTTCCC | 32 | <50 |
| | 151(+192) | TACGGTGAGCGCTTCCCCG | 33 | <50 |
| | 171(+192) | TGAGAACTTCAAACTGAAG | 34 | <50 |
| | 173(+192) | AGAACTTCAAACTGAAGCA | 35 | <50 |
| | I79(+192) | TCAAACTGAAGCACTACGG | 36 | <50 |

*represents abase sequence described as SEQ ID NO: 1 from a designated position to 21^{th} nucleotide.

Referring to FIG. 3, the siRNA includes 18 binding energy patterns. The correlation between the 18 binding energy patterns of siRNA having a specific base sequence obtained from the step (a) and the inhibition efficiency of gene expression is determined depending on how the 18 binding energy patterns are divided into sections to grasp the entire pattern of the binding energy. As a result, the present inventors calculated the mean of each binding energy pattern from the 1^{st} through 18^{th} positions in 140 experimental data sets for siRNA inhibition of gene expression obtained from (a), and then showed a graph having an axis x from the 1^{st} to 18^{th} positions and an axis y of the binding energy (−ΔG) as shown in FIG. 5.

The present inventors set sections to have a phenomenon where a difference of the mean binding energy between one section and its adjacent section is most largely reversed between effective siRNA (over 90% gene inhibition) and ineffective siRNA (below 50% gene inhibition). That is, when the 18 binding energy locations are divided into a plurality of sections, preferably four sections A, B, C and D, and each mean energy is defined E_{A}, E_{B}, E_{C} and E_{D}, and sections are set such that a difference of the mean binding energy in each section of the effective siRNA and the ineffective siRNA, that is, E_{A}-E_{B}, E_{B}-E_{C}, E_{C}-E_{D}, is the farthest from 0 to show the largest change.

To do so, the experimental data of siRNA gene expression inhibition are divided into an effective group and an ineffective group. A null hypothesis that there is no difference between the two groups in the 1^{st}˜18^{th} binding energy locations was verified through a t-test. That is, the binding energy location having a p-value of less than 0.05 has a difference of the binding energy around a significance level of 5% in the two groups. FIG. 6 is a graph illustrating a result in an axis x of the binding energy location and an axis y of the p-value, and FIG. 7 is a graph with a smooth curved line in an axis x of the binding energy location and an axis y of the t-value obtained by the following Equation 1.

$$( t \text{-} value ) = \frac{\overline{X} - \overline{Y}}{\sqrt{\frac{{S}_{x}}{{N}_{x}} + \frac{{S}_{y}}{{N}_{y}}}} \qquad [ Equation 1 ]$$

herein,
X̅: the mean binding energy of the effective group;
Y̅: the mean binding energy of the ineffective group;
Sₓ: the distribution of the effective group;
S_{y}: the distribution of the ineffective group;
Nₓ: the number of variation of the effective group;
N_{y}: the number of variation of the ineffective group.

Three kinds of data sets are used in the preferred embodiment of the present invention. The two data sets extracted from Khvorova's paper include experimental results of gene inhibition on pGL3 and hCyPB that are classified into the efficient group (over 90% inhibition) and the inefficient group (below 50% inhibition). The one data set extracted from Amarzguioui's paper includes experimental results on various kinds of genes (AA) that are compositely classified into the effective group (over 70% inhibition) and the ineffective group (below 70% inhibition). Khvorova's paper includes 40 effective results and 20 ineffective results on gene firefly luciferase (pGL3), and 13 efficient results and 21 inefficient results on human cyclophilin (hCyPB). Amarzguioui's paper includes 21 effective results and 25 ineffective results on various kinds of genes (AA).

The present inventors noticed that the t-value change type of the three data sets was shown in the same pattern as shown in FIG. 7. As it was expected that the division of the effective and ineffective groups in the data set obtained from Amarzguioui's paper is more ambiguous than that in the rest data sets, the data set obtained from Amarzguioui's paper was shown to have a smaller change width of the t-value than that of the rest data sets. It means that there is a specific division of the binding energy pattern between the effective siRNA and the ineffective siRNA.

The t-value has a maximum or minimum value, or the p-value becomes close to 0 where a difference of the binding energy between the effective siRNA group and the ineffective siRNA group is extremely large. That is, if a neighboring area with this part as the center is set as one section, the deviation of the binding energy between the neighboring sections can be maximized. Even though the t-value has a maximum or minimum value, where the deviation of the maximum and minimum values of the t-value is not large, that is, the p-value is not considered as being discriminative, and they may be excluded in designation of sections.

In the preferred embodiment of the present invention, locations which are the center of the section are designated using the p-value of FIG. 6. Here, the following standards are applied.
\textcircled{1} where the p-value of one or more of the two data sets of Khovorova is 0.1 or less
\textcircled{2} where all of the two data sets of Khovorova are 0.4 or less

The location suitable for standard \textcircled{1} and \textcircled{2} includes the 1^{st} binding energy location, 5˜6^{th} binding energy location, 14^{th} binding energy location and 17˜18^{th} binding energy location.

Hereinafter, only the two data sets of Khovorova are used because the group division standard in the data sets of Amarzoguioui is different from that of the two data sets of Khovorova, and also performance is to be tested after a method for evaluating the efficiency of siRNA according to the present invention is established.

Next, a section is determined with the above four locations as the center. The base of the determination of the section is to maximize the change of the difference between the mean binding energy of the determined section and the binding energy of the other adjacent section. Preferably, the subsequent process can be divided into the following two cases.
(1) when the process is set to be continuously performed without any vacant space between the adjacent sections
(2) when the process is set to be discontinuously performed with a vacant space between the adjacent sections

The above two cases have both merits and demerits. The case (1) degrades the prediction due to a partially undistinguished section although the status of all binding energy can be examined. On the other hand, the case (2) cannot evaluate the location although the undistinguished section is excluded to maximize the prediction.

Preferably, the section (1) is set as follows.

The section (a) is divided into four sections A, B, C and D to include four locations set based on the standards \textcircled{1} and \textcircled{2} respectively and also include locations of all binding energy without invading regions of other locations, thereby obtaining 20 combinations as shown in Table 2.

TABLE 2

| Section A | Section B | Section C | Section D |
|---|---|---|---|
| 1~2 | 3~7 | 8~14 | 15~18 |
| 1~2 | 3~8 | 9~14 | 15~18 |
| 1~2 | 3~9 | 10~14 | 15~18 |
| 1~2 | 3~10 | 11~14 | 15~18 |
| 1~2 | 3~11 | 12~14 | 15~18 |
| 1~2 | 3~7 | 8~15 | 16~18 |
| 1~2 | 3~8 | 9~15 | 16~18 |
| 1~2 | 3~9 | 10~15 | 16~18 |
| 1~2 | 3~10 | 11~15 | 16~18 |
| 1~2 | 3~11 | 12~15 | 16~18 |
| 1~3 | 4~7 | 8~14 | 15~18 |
| 1~3 | 4~8 | 9~14 | 15~18 |
| 1~3 | 4~9 | 10~14 | 15~18 |
| 1~3 | 4~10 | 11~14 | 15~18 |
| 1~3 | 4~11 | 12~14 | 15~18 |
| 1~3 | 4~7 | 8~15 | 16~18 |
| 1~3 | 4~8 | 9~15 | 16~18 |
| 1~3 | 4~9 | 10~15 | 16~18 |
| 1~3 | 4~10 | 11~15 | 16~18 |
| 1~3 | 4~11 | 12~15 | 16~18 |

Here, the number of effective siRNAs is N_{f} and the number of ineffective siRNAs is Nₙ, the efficiency is i (‘f’ in case of siRNA of the effective group, ‘n’ in case of siRNA of the ineffective group). The mean binding energy per one binding energy that the jth (to have a number of 1˜N_{f} or 1˜Nₙ as a value) siRNA has in a section k (one of A, B, C and D) is defined as Eᵢⱼₖ. That is, the mean energy per one binding energy is represented as E_{f3B} in the section B of the 3^{rd} siRNA of the effective group. Each Eᵢⱼₖ is obtained using experimental data.

The variation of the mean binding energy which becomes a representative among sections A˜B(E_{i(A-B)}), B˜C(E_{i(B-C)}), C˜D(E_{i(C-D)}) is obtained using each Eᵢⱼₖ depending on the following Equation 2.

$${E}_{i ( A - B )} = {E}_{iA} - {E}_{iB} = \frac{1}{{N}_{i}} {\sum }_{j} ( {E}_{ijA} - {E}_{ijB} ) \qquad [ Equation 2 ]$$

E_{i(B-C)} and E_{i(C-D)} may be obtained using the Equation 2. Here, E_{f(A-B)} is a value that represents binding energy per one binding energy location in the sections A and B of siRNAs of the effective group, and E_{n(A-B)} is that of the ineffective group. That is, if a section is taken to increase an absolute value of E_{f(A-B)}−E_{n(A-B)}, a difference of the mean binding energy between the effective siRNA group and the ineffective siRNA group in the sections A and B becomes larger. As a result, a section can be selected using the above-described characteristic. The same goes for B˜C and C˜D. The present inventors selected only combinations of sections having an absolute value of 0.1 or more in E_{f(A-B)}−E_{n(A-B)}, E_{f(B-C)}−E_{n(B-C)} and E_{f(C-D)}−E_{n(C-D)}. In the preferred embodiment of the present invention, four sections are selected, and Table 3 shows information on the selected sections.

TABLE 3

| Section A | Section B | Section C | Section D |
|---|---|---|---|
| 1~2 | 3~7 | 8~15 | 16~18 |
| 1~2 | 3~8 | 9~15 | 16~18 |
| 1~3 | 4~7 | 8~15 | 16~18 |
| 1~3 | 4~8 | 9~15 | 16~18 |

The t-test is performed among E_{f(A-B)} and E_{n(A-B)}, E_{f(B-C)} and E_{n(B-C)}, and E_{f(C-D)} and E_{n(C-D)} in the selected four sections to obtain a t-value and a p-value. Through this process, one section for distinguishing the effective siRNA group and the ineffective siRNA group is determined in p-value<0.05, t-value>2 of all sections of gene hCyPB, pGL3. The sections are A(1˜2), B(3˜7), C(8˜15) and D(16˜18), and FIG. 8 shows information on these sections.

Preferably, the section (2) is set as follows:

The same procedure of the section (1) is basically repeated, except that a different method is used to set a width of the section since the sections are allowed to be discontinuous and overlapped with each other. Table 4 shows combinations of all sections in the 2 binding energy location including 4 binding energy locations set based on the standards \textcircled{1} and \textcircled{2}.

TABLE 4

| | | | | | | | | | |
|---|---|---|---|---|---|---|---|---|---|
| Section A | 1 | 1~2 | 1~3 | | | | | | |
| Section B | 3~6 | 4~6 | 5~6 | 3~7 | 4~7 | 5~7 | 3~8 | 4~8 | 5~8 |
| Section C | 12~14 | 13~14 | 14 | 12~15 | 13~15 | 14~15 | 12~16 | 13~16 | 14~16 |
| Section D | 15~18 | 16~18 | 17~18 | | | | | | |

If one of the sections A, B, C and D is selected in Table 4, a combination of the necessary section is performed. As a result, 729 (=3×9×9×3) kinds of combinations are possible. Since it is almost impossible to select only one combination of one section through the method of the equation 2 and the t-test in the 729 combinations, a new variable R (abbreviation of robustness) is preferably introduced. R is a figure that represents how many bonding energies are located in the section excluding 4 bonding energies set by the standards \textcircled{1} and \textcircled{2}. For examples, if the section A is set as 1˜2 and the section B is set as 4˜7, the R value of the section A is 1 and the R value of the section B is 2. When the R value of the two sections like (1) E_{f(A-B)} of the section A(1˜2) and the section B(4˜7) is under consideration, each R value of the two sections are added so that the R value in the section A˜B is set as 3.

The Eᵢⱼₖ mentioned in (1) is respectively obtained in all combinations of the sections A, B, C and D shown in Table 4. The values E_{i(A-B)}, E_{i(B-C)} and E_{i(C-D)} calculated from the equation 2 are obtained in all combinations through Table 4, and the t-test is performed to obtain respective t-value and p-value. Here, the above-mentioned R value is applied. FIG. 9 is a graph illustrating a ratio of combination with p-values of 0.05 less in total combinations having a specific R value of the sections A˜B, B˜C and C˜D. As the R value becomes larger, the p-value tends to decrease. As a result, the R value before radical decrease of the p-value is calculated to obtain a section including the largest range having a desired p-value. Referring to FIG. 9, when the R value is 3 or 4 or less, the ratio of the section of p-value<0.05 is shown to be higher. Therefore, only the sections having R=3 or 4 are included in proposed sections in the preferred embodiment of the present invention.

The final sections are determined through the R value and the t-test results. Since the R value is required to be 3 or 4 in the two sections, two binding energy locations are added in the sections B and C where a section is added in both sides, and one binding energy location is added in the sections A and D where a section is added in one side. As a result, R=3 in A˜B, R=4 in B˜C and R=3 in C˜D. After all combinations of sections satisfying this condition are made, the t-test is performed on these combinations to select one section combination having an extremely low p-value.

The selected sections are A(1˜2), B(3˜6), C(14˜16) and D(16˜18). Table 5 shows information on these sections.

TABLE 5

| | | Section A-B | Section B-C | Section C-D |
|---|---|---|---|---|
| | | 1~2 | 3~6 | 14~16 |
| | | 3~6 | 14~16 | 16~18 |
| hCyPB | t-value | 3.175553 | −3.4246 | 5.915552 |
| | p-value | 0.00165 | 0.000853 | 0.000001 |
| pGL3 | t-value | 2.68004 | −2.32939 | 3.217273 |
| | p-value | 0.004783 | 0.011671 | 0.001059 |
| AA | t-value | 1.887835 | −0.89566 | 1.266718 |
| | p-value | 0.032827 | 0.18765 | 0.10596 |

In the preferred embodiment of the present invention, the two sections set through (1) and (2) (see FIG. 10) are selected by distinguishing a relative binding energy pattern with the adjacent section. However, since there is a sufficient difference of the binding energy between non-adjacent sections, the t-test is performed on six combinations of A-B, B-C, C-D, A-C, A-D and B-D obtained by the difference of the four sections A, B, C and D. Table 6 shows the t-test results.

TABLE 6

| Section A | Section B | Section C | Section D | | | | |
|---|---|---|---|---|---|---|---|
| 1~2 | 3~7 | 8~15 | 16~18 | | | | |
| | | Section | Section | Section | Section | Section | Section |
| | | A-B | B-C | C-D | A-C | A-D | B-D |
| hCyPB | t-value | 3.15303 | −2.25399 | 3.27599 | 1.38792 | 5.40182 | 1.00611 |
| | p-value | 0.00175 | 0.01559 | 0.00127 | 0.08737 | 0.00000 | 0.16095 |
| pGL3 | t-value | 2.42243 | −2.40223 | 2.13573 | 0.42633 | 2.31082 | 0.15585 |
| | p-value | 0.00928 | 0.00976 | 0.01847 | 0.33572 | 0.01221 | 0.42834 |
| AA | t-value | 1.87483 | −1.02960 | 1.09863 | 1.41229 | 1.94585 | 0.22186 |
| | p-value | 0.03373 | 0.15441 | 0.13895 | 0.08245 | 0.02904 | 0.41273 |
| Section A | Section B | Section C | Section D | | | | |
| 1~2 | 3~6 | 14~16 | 16~18 | | | | |
| | | | Section | Section | Section | Section | Section |
| | | | A-B | B-C | A-C | A-D | B-D |
| hCyPB | t-value | 3.16461 | −3.42274 | 5.92078 | 0.65134 | 5.40182 | 0.82726 |
| | p-value | 0.00340 | 0.00172 | 0.00000 | 0.51948 | 0.00001 | 0.41421 |
| pGL3 | t-value | 2.69174 | −2.32867 | 3.20424 | 0.17064 | 2.31082 | 0.32109 |
| | p-value | 0.00464 | 0.01169 | 0.00110 | 0.43255 | 0.01221 | 0.37465 |
| AA | t-value | 1.89671 | −0.91889 | 1.27660 | 1.29998 | 1.94585 | 0.16337 |
| | p-value | 0.03222 | 0.18158 | 0.10422 | 0.10019 | 0.02904 | 0.43549 |

As shown in Table 6, there is no big difference in the sections A-C and B-D. The combination of A-D satisfies the condition of p-value<0.05 in the non-adjacent section. Here, the fact that a difference of binding energy between the section A of 5′ end and the section B of 3′ end affects the efficiency of siRNA has been well known in other experimental results (Schwarz, D. S., Hutvagner, G, Du, T., Xu, Z., A ronin, N., Zamore, P. D., Cell, 115(2), 199-20, 2003).

The present inventors used the collected experimental data and selected sections for calculating the relative binding energy of unknown siRNA. For establishing a scoring system, the two data sets extracted from the Khvorova's paper, that is the experimental results on firefly luciferase (pGL3) and human cyclophilin (hCyPB) are included in the collected data to obtain a larger data set. One data set extracted from the Amarzguioui's paper obtained by dividing the set on a basis of 70% inhibition efficiency of gene expression was excluded in the data for establishing the scoring system since the classification standard was different from that of the data of the Khvorova's paper that regarded 90% or more as effective and 50% or less as ineffective. The obtained data were classified into the effective group (inhibition efficiency of gene expression of 90% or more: functional or f) and the ineffective group (inhibition efficiency of gene expression of 50% less; nonfunctional or n).

The obtained data are divided into the sections obtained by the above-described process to obtain E_{i(A-B)}, E_{i(B-C)}, E_{i(C-D)} and E_{i(A-D)} from the equation 2. These values mean energy values obtained by averaging values on difference of the average energy in each group. In this process, each value has distribution values which are S_{i(A-B)}, S_{i(B-C)}, S_{i(C-D)} and S_{i(A-D)}. The number of siRNA experimental data is defined as Nᵢ. Table 7 shows values E_{i(A-B)}, E_{i(B-C)}, E_{i(C-D)}, E_{i(A-D)}, values S_{i(A-B)}, S_{i(B-C)}, S_{i(C-D)}, S_{i(A-D)}, Nᵢ, and t-values and p-values through the t-test.

TABLE 7

| Section A | Section B | Section C | Section D | | |
|---|---|---|---|---|---|
| 1~2 | 3~7 | 8~15 | 16~18 | | |
| | | Section A-B | Section B-C | Section C-D | Section A-D |
| | mean(Ef) | 0.18 | −0.15 | 0.18 | 0.22 |
| effective | distribution(Sf) | 0.55 | 0.28 | 0.41 | 0.32 |
| Nf = 53 | Standard | 0.74 | 0.53 | 0.64 | 0.57 |
| | deviation | | | | |
| | Nf | 53 | 53 | 53 | 53 |
| | mean(Ef) | −0.42 | 0.25 | −0.28 | −0.45 |
| ineffective | distribution(Sf) | 0.49 | 0.43 | 0.4 | 0.53 |
| Nn = 41 | Standard | 0.7 | 0.65 | 0.63 | 0.73 |
| | deviation | | | | |
| | Nn | 41 | 41 | 41 | 41 |
| | T | 4.026342 | −3.16981 | 3.489798 | 4.826898 |
| | P | 0.000058 | 0.001036 | 0.000372 | 0.000003 |
| Section A | Section B | Section C | Section D | | |
| 1~2 | 3~6 | 14~16 | 16~18 | | |
| | | Section A-B | Section B-C | Section C-D | Section A-D |
| | mean(Ef) | 0.2 | −0.21 | 0.23 | 0.22 |
| effective | distribution(Sf) | 0.56 | 0.57 | 0.34 | 0.32 |
| Nf = 53 | Standard | 0.75 | 0.75 | 0.59 | 0.57 |
| | deviation | | | | |
| | Nf | 53 | 53 | 53 | 53 |
| | mean(Ef) | −0.42 | 0.3 | −0.33 | −0.45 |
| ineffective | distribution(Sf) | 0.47 | 0.45 | 0.21 | 0.53 |
| Nn = 41 | Standard | 0.69 | 0.67 | 0.46 | 0.73 |
| | deviation | | | | |
| | Nn | 41 | 41 | 41 | 41 |
| | T | 4.166805 | −3.49839 | 5.207057 | 4.826898 |
| | P | 0.000035 | 0.000362 | 0.000001 | 0.000003 |

As shown in Table 7, since the data set is p-value<0.05 in all sections, it can be used in the scoring system for dividing the effective siRNA and the ineffective siRNA.

If the mean binding energy difference between the sections A and B of a specific siRNA in the effective siRNA group is X_{f(A-B)}, X ranges according to the equation 3 in the significance level of p-value<0.05.

$${E}_{f ( A - B )} - 1.96 \sqrt{\frac{{S}_{f ( A - B )}}{{N}_{f}}} < {X}_{f ( A - B )} < {E}_{f ( A - B )} + 1.96 \sqrt{\frac{{S}_{f ( A - B )}}{{N}_{f}}} \qquad [ Equation 3 ]$$

The equation 3 can be applied to all of X_{i(A-B)}, X_{i(B-C)}, X_{i(C-D)} and X_{i(A-D)}, and also each range of values X_{i(A-B)}, X_{i(B-C)}, X_{i(C-D)} and X_{i(A-D)} can be obtained as shown in FIG. 11.

The efficiency of unknown siRNA is scored through the relative binding energy pattern under consideration of the results by:

1) obtaining the average binding energy values, that is, X_{(A-B)}, X_{(B-C)}, X_{(C-D)} and X_{(A-D)}, in the sections A-B, B-C, C-D and A-D of unknown siRNA

2) determining which range the value of X_{(A-B)} belongs to and give a score as follows:
i)

$$if {E}_{f ( A - B )} - 1.96 \sqrt{\frac{{S}_{f ( A - B )}}{{N}_{f}}} < {X}_{( A - B )} < {E}_{f ( A - B )} + 1.96 \sqrt{\frac{{S}_{f ( A - B )}}{{N}_{f}}} , \\ 10$$

points are given;
ii)

$$if {E}_{n ( A - B )} - 1.96 \sqrt{\frac{{S}_{n ( A - B )}}{{N}_{n}}} < {X}_{( A - B )} < {E}_{n ( A - B )} + 1.96 \sqrt{\frac{{S}_{n ( A - B )}}{{N}_{n}}} , \\ 0 point is given ;$$

iii) when the range does not belong to i) or ii), 5 points are given.

In the same way, scores are given to X_{(B-C)}, X_{(C-D)} and X_{(A-D)}.

Each score is defined as Y_{(A-B)}, Y_{(B-C)}, Y_{(C-D)} and Y_{(A-D)}.

Referring to FIG. 11, in the continuous section, if −0.02<X_{(A-B)}<0.38, −0.29<X_{(B-C)}<−0.01, 0.00<X_{(C-D)}<0.35, 0.07<X_{(A-D)}<0.37, then Y_{(A-B)}, Y_{(B-C)}, Y_{(C-D)} and Y_{(A-D)} are individually given 10 points, and if −0.63<X_{(A-B)}<−0.21, 0.05<X_{(B-C)}<0.44, −0.47<X_{(C-D)}<−0.09, −0.67<X_{(A-D)}<−0.23, then Y_{(A-B)}, Y_{(B-C)}, Y_{(C-D)} and Y_{(A-D)} are individually given 0 point, and Y_{(A-B)}, Y_{(B-C)}, Y_{(C-D)} and Y_{(A-D)} are individually given 5 points when X_{(A-B)}, X_{(B-C)}, X_{(C-D)} and X_{(A-D)} do not belong to said ranges.

In the discontinuous section, if 0.00<X_{(A-B)}<0.40, −0.41<X_{(B-C)}<−0.01, 0.07<X_{(C-D)}<0.39, 0.07<X_{(A-D)}<0.37, then Y_{(A-B)}, Y_{(B-C)}, Y_{(C-D)} and Y_{(A-D)} are individually given 10 points, and if −0.63<X_{(A-B)}<−0.21, 0.10<X_{(B-C)}<0.51, −0.47<X_{(C-D)}<−0.19, −0.67<X_{(A-D)}<−0.23, then Y_{(A-B)}, Y_{(B-C)}, Y_{(C-D)} and Y_{(A-D)} are individually given 0 point, and Y_{(A-B)}, Y_{(B-C)}, Y_{(C-D)} and Y_{(A-D)} are individually given 5 points when X_{(A-B)}, X_{(B-C)}, X_{(C-D)} and X_{(A-D)} do not belong to said ranges.

3) when weighting factors of Y_{(A-B)}, Y_{(B-C)}, Y_{(C-D)} and Y_{(A-D)} are defined as W_{(A-B)}, W_{(B-C)}, W_{(C-D)} and W_{(A-D)}, the score Y of the relative binding energy pattern is converted based on full mark 100 points using the equation 4:

$$Y = \frac{\begin{matrix}{W}_{( A - B )} {Y}_{( A - B )} + {W}_{( B - C )} {Y}_{( B - C )} + \\ {W}_{( C - D )} {Y}_{( C - D )} + {W}_{( A - D )} {Y}_{( A - D )}\end{matrix}}{10 ( {W}_{( A - B )} + {W}_{( B - C )} + {W}_{( C - D )} + {W}_{( A - D )} )} \times 100 \qquad [ Equation 4 ]$$

The binding energy pattern of siRNA is scored depending on how the weighting factors W_{(A-B)}, W_{(B-C)}, W_{(C-D)} and W_{(A-D)} in each section are set. In order to optimize the combination of the weighting factors, the t-value between the effective siRNA group and the ineffective siRNA is examined as each weighting factor is increased from 0 to 1 by 0.01. FIG. 12 shows distribution of combinations depending on each weighting factor value among the upper 100 t-values which are arranged in a descending order. Referring to the distribution of FIG. 12, a location for maximizing a t-value, that is, a location for maximizing a difference of the binding energy variation between the effective siRNA group and the ineffective siRNA group can be found. The combination of W_{(A-B)}, W_{(B-C)}, W_{(C-D)} and W_{(A-D)} for maximizing the t-value between the two groups is ranging from 0.90 to 1.00, 0.2 to 0.4, 0.2 to 0.3 and 0.7 to 0.9, preferably, 1.00, 0.37, 0.20, 0.90 in the continuous section, and ranging from 0.5 to 0.7, 0.3 to 0.5, 0.3 to 0.5 and 0.9 to 1.0, preferably, 0.65, 0.48, 0.48 and 0.90 in the discontinuous section. If it is set beyond a threshold value in each case, the t-value is rapidly decreased even to insignificant level for discriminating in the scoring method.

Finally, the present inventors considered how the relative binding energy pattern can be combined with other factors (GC content, Tₘ, absolute scores of binding energy, homology with other mRNA, secondary structure of RNA) to obtain a system for predicting the overall efficiency of siRNA. The following linear equation basically the same way of scoring the relative binding energy pattern is used as a scoring method.

$${S}_{t} = {\sum }_{i} {W}_{i} {S}_{i}$$

If the score given to each factor is defined as Zᵢ(Z₁, Z₂, Z₃, . . . , Zₙ), the full mark of each factor is defined as Mᵢ(M₁, M₂, M₃, . . . , Mₙ), and the efficiency of each factor, that is, the weighting factor of each score is defined as Wᵢ(W₁, W₂, W₃, . . . , Wₙ), then the score Z that represents the efficiency of siRNA can be expressed based on full mark 100 points according to the equation 5:

$$Z = 100 \times \frac{{\sum }_{i} {W}_{i} \frac{{Z}_{i}}{{M}_{i}}}{{\sum }_{i} {W}_{i}} \qquad [ Equation 5 ]$$

wherein i is an integer ranging from 1 to n, Zᵢ comprising various factors for affecting inhibition of target mRNA includes the relative binding energy as an essential factor and one or more factors selected from the group comprising the number of A/U in 5 bases of 3′-end, the presence of G/C at 1^{st} position, the presence of A/U at 19^{th} position, the content of G/C, Tₘ, secondary structure of RNA, the homology with other mRNA and the like as an optional factor. The optional factors are not necessarily included in allotting the Z value but factors for inducing better prediction with the relative binding energy can be included without limitation. Also, there is no specific limitation in combination of factors. In the preferred embodiment of the present invention, the following factors are selected as Zᵢ: Z₁—the score (Y) of the relative binding energy, Z₂—the number of A/U in 5 bases of 3′-end, Z₃—the presence of G/C at 1^{st} position, Z₄—the presence of A/U at 19^{th} position, Z₅—the score of G/C content. The respective value of Mᵢ is as follows: M₁=100, M₂=5, M₃=1, M₄=1, M₅=10.

In the preferred embodiment of the present invention, Z₁ is the calculated score Y, Z₂ is the number of A/U in 5 bases of 3′-end, Z₃ is 1 when the base of 5′ end is G/C or 0 when it isn't, Z₄ is 1 when the base of 3′ end is A/U or 0 when it isn't, and Z₅ is 10 when the content of G/C ranges from 36 to 53% and 0 when it does not belong within the range.

FIG. 13 is a graph for optimizing the weighting factor Wᵢ on each score in the same way of the scoring the relative binding energy pattern as in FIG. 12. The combination of W₁, W₂, W₃, W₄ and W₅ optimized through this process ranges from 0.9 to 1.0, from 0.0 to 0.2, from 0.1 to 0.3 and from 0.0 to 0.2, preferably, 0.90, 0.07, 0.15, 0.19 and 0.11.

The Z value obtained through the above process can be an index for distinguishing which relative binding energy pattern unknown siRNA has. As a result, only the analysis of the base sequence enables evaluation of the binding energy, thereby maximizing the design and production efficiency of siRNA.

According to the present invention, it is possible to predict the inhibition efficiency of unknown siRNA to target mRNA. As a result, the expression of target mRNA can be effectively inhibited by applying a selected siRNA having an excellent inhibition efficiency, preferably a selected siRNA having a Z value within upper 10% to the target mRNA using the above-described method. The above numerical value can be any value and may be flexibly applied depending on sample size of a candidate siRNA group, experimental conditions and the like.

### BRIEF DESCRIPTION OF THE DRAWINGS

FIG. 1 is a diagram illustrating inhibition efficiency of gene expression of siRNA changes depending on combination patterns of RISC enzyme.

FIG. 2 is a diagram illustrating a method for scoring the relationship between the inhibition efficiency of gene expression and the binding energy of siRNA.

FIG. 3 is a diagram illustrating binding energy distribution of binding energy of siRNA in INN-HB nearest neighbor model.

FIG. 4 illustrates binding energy values in INN-HB nearest neighbor model.

FIG. 5 is a graph illustrating the mean of the binding energy in each location of collected siRNA data:

axis X; from the 1^{st} to 18^{th} positions,

axis Y; mean of the binding energy (−ΔG),

solid line; when the inhibition efficiency of gene expression is 90% or more,

dotted line; when the inhibition efficiency of gene expression is below 50%.

FIG. 6 is a graph illustrating t-test result of the binding energy in each location of collected siRNA data:

axis X; from the 1^{st} to 18^{th} positions, axis Y; p-value,

solid line; pGL3 gene, dotted line; hCyPB gene

dash-dot line; complex gene extracted from Amarzguioui's paper.

FIG. 7 is a graph illustrating t-test result of the binding energy in each location of collected siRNA data:

axis X; from the 1^{st} to 18^{th} positions, axis Y; t-value,

solid line; pGL3 gene, dotted line; hCyPB gene

dash-dot line; complex gene extracted from Amarzguioui's paper.

FIG. 8 is a graph illustrating various information on sections A(1˜2), B(3˜7), C(8˜15) and D(16˜18) obtained by analyzing binding energy data through the process (1).

FIG. 9 is a graph illustrating ratio distribution where the p-value is less than 0.05 among the combination of A-B, B-C and C-D having a specific R value.

FIG. 10 is a diagram illustrating a section selected through the processes (1) and (2).

FIG. 11 illustrates a graph (A) that shows a reliable section of a relative difference between the mean binding energy of ineffective siRNA and effective siRNA in the sections A˜B, B˜C, C˜D and A˜D selected through the process (1) and a graph (B) that shows a reliable section between a relative difference of the mean binding energy of ineffective siRNA and effective siRNA in the sections A˜B, B˜C, C˜D and A˜D selected through the process (2).

FIG. 12 is a graph illustrating the relationship between weighting factor and the t-value in the score of relative binding energy pattern, wherein the combination of weighting factors are arranged in a descending order depending on the t-value to show the number of the weighting factors of the upper 100 combinations in each section. Here, A is distribution of the weighting factors in the continuous section, and B is distribution of weighting factors in the discontinuous section.

FIG. 13 shows a graph for optimizing the weighting factor Wᵢ on each score in the same way of scoring the relative binding energy pattern as shown in FIG. 12.

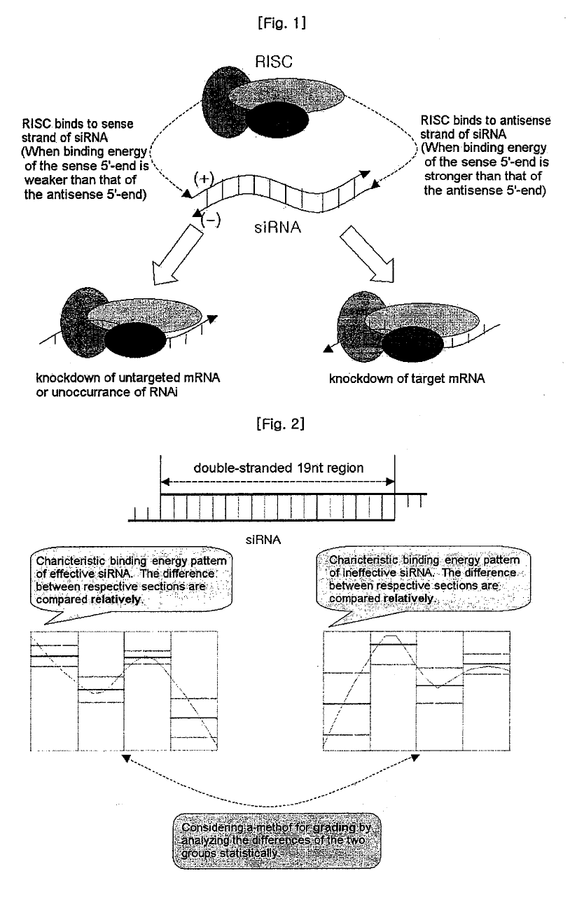

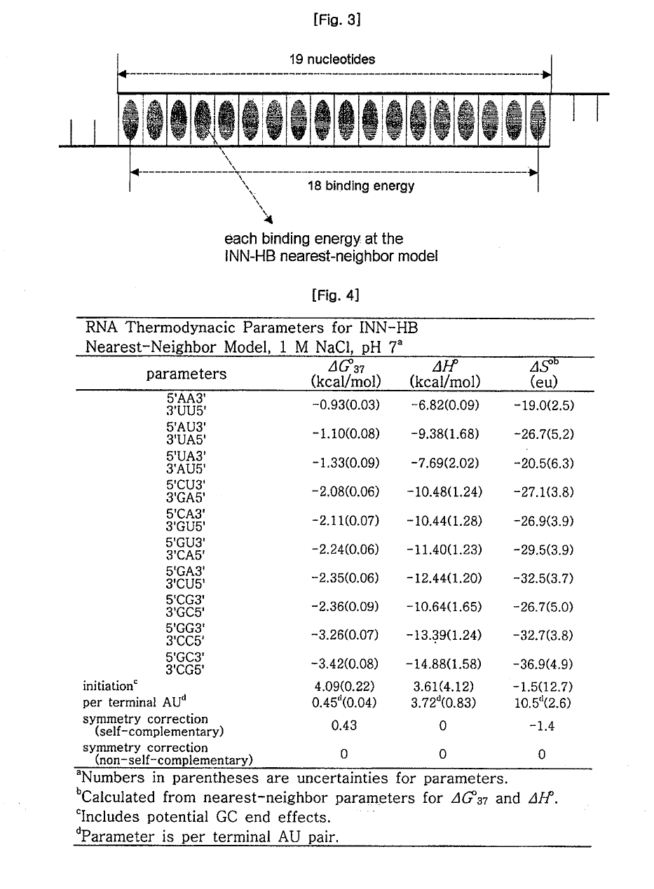

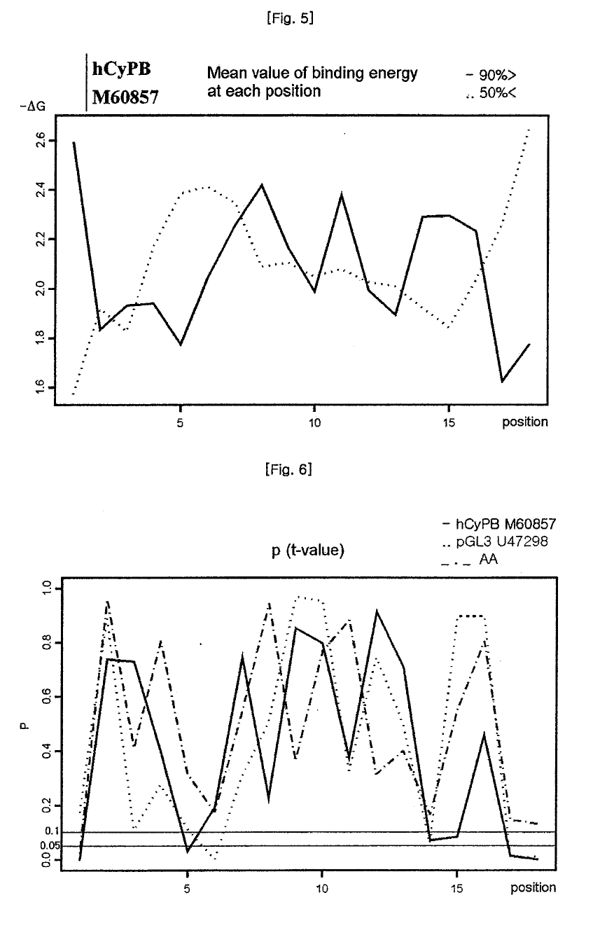

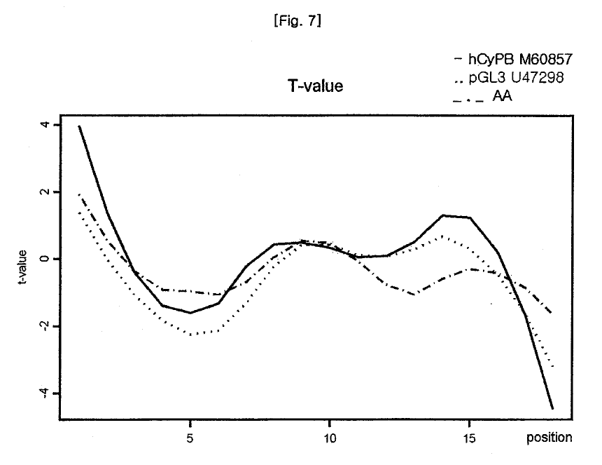

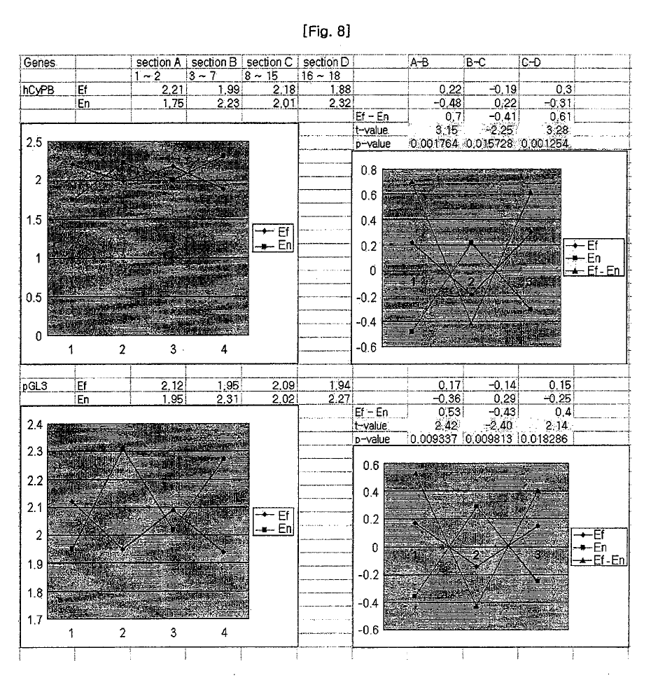

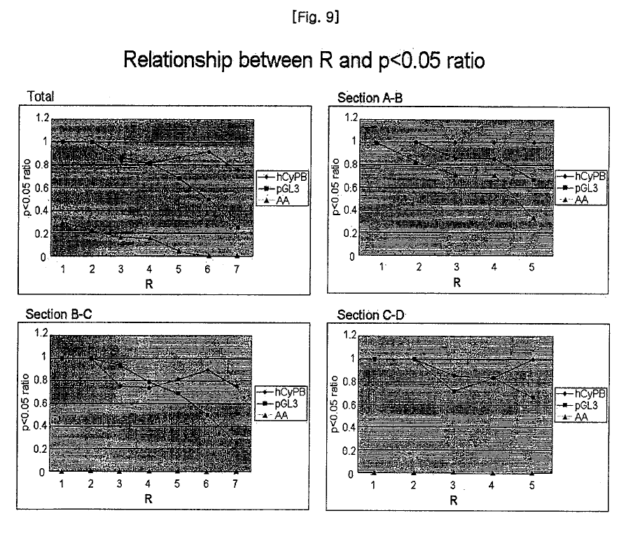

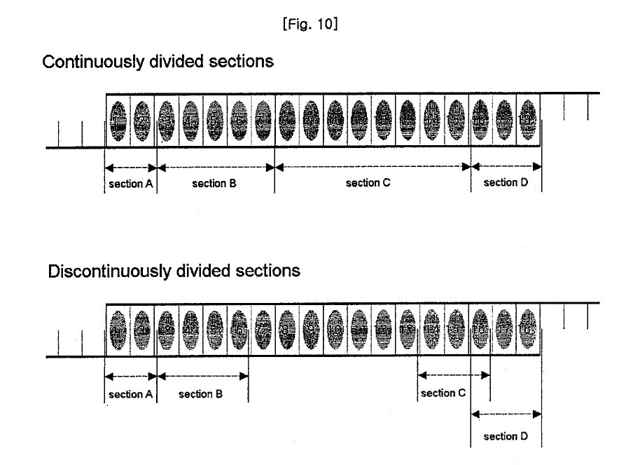

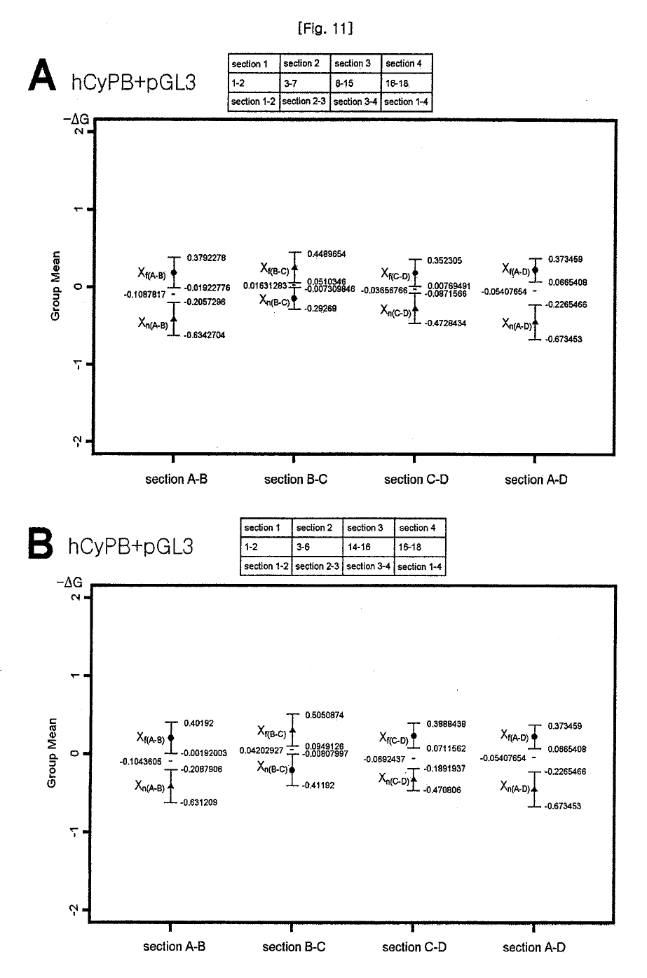

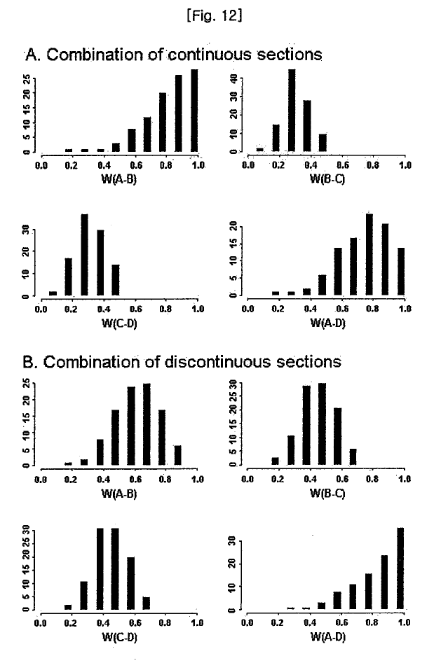

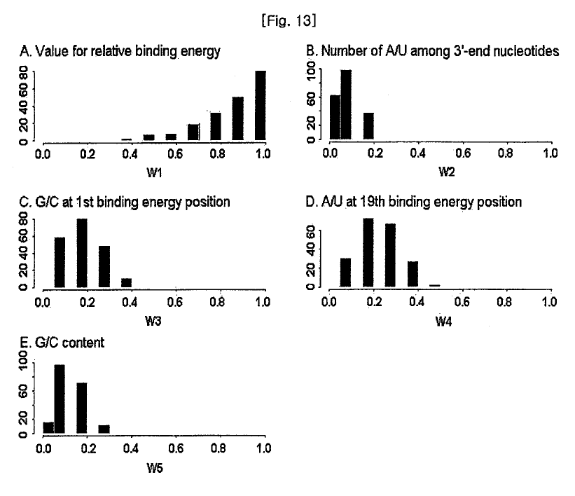

### PREFERRED EMBODIMENTS

The present invention will be described in detail by referring to examples below, which are not intended to limit the present invention.

### Example 1

### Comparison with Conventional Method of siRNA Design

In order to test the performance of the siRNA design optimizing method using the relative binding energy pattern according to the present invention, the siRNA design optimizing method was compared with the scoring method of the siRNA design disclosed in Patent No. WO2004/045543 (Functional and Hyperfunctional siRNA, published on Jun. 3, 2004). The scoring method of siRNA efficiency disclosed in many algorithms of the Patent No. WO2004/045543 was performed according to the following equation 6:

Relative functionality of siRNA=−(GC/3)+(AU₁₅₋₁₉)−(Tm_{20° C.})*3−(G₁₃)*3−(C₁₉)+(A₁₉)*2+(A₃)+(U₁₀)+(A₁₃)−(U₅)−(A₁₁) [Equation 6]

Of the three data sets obtained from Khvorova's paper and Amarzguioui's paper, one data set extracted from the Amarzguioui's paper except the two data sets extracted from the Khvorova's paper used in scoring the relative binding energy pattern was used as a test set to compare prediction of two scoring methods. First, each score of siRNA included in the effective/ineffective groups was calculated using the two scoring methods. Through LDA (Linear discriminant analysis) and QDA (Quadratic discriminant analysis), decision on whether a random siRNA was effective or ineffective was calculated. Preferably, the above value can be obtained using a statistical program R (http://www.R-project.org) ([1] Richard A. Becker, John M. Chambers, and Allan R. Wilks. The New S Language. Chapman & Hall, London, 1988; [2] John M. Chambers and Trevor J. Hastie. Statistical Models in S. Chapman & Hall, London, 1992; [3] John M. Chambers. Programming with Data. Springer, New York, 1998. ISBN 0-387-98503-4; [4] William N. Venables and Brian D. Ripley. Modern Applied Statistics with S. Fourth Edition. Springer, 2002. ISBN 0-387-95457-0; [5] William N. Venables and Brian D. Ripley. S Programming. Springer, 2000. ISBN 0-387-98966-8; [6] Deborah Nolan and Terry Speed. Stat Labs Mathematical Statistics Through Applications. Springer Texts in Statistics. Springer, 2000. ISBN 0-387-98974-9; [7] Jose C. Pinheiro and Douglas M. Bates. Mixed-Effects Models in S and S-Plus. Springer, 2000. ISBN 0-387-98957-0; [8] Frank E. Harrell. Regression Modeling Strategies, with Applications to Linear Models, Survival Analysis and Logistic Regression. Springer, 2001. ISBN 0-387-95232-2; [9] Manuel Castejon Limas, Joaquin Ordieres Mere, Fco. Javier de Cos Juez, and Fco. Javier Martinez de Pison Ascacibar. Control de Calidad. Metodologia para el analisis previo a la modelizacion de datos en procesos industrials. Fundamentos teoricos y aplicaciones con R. Servicio de Publicaciones de la Universidad de la Rioja, 2001. ISBN 84-95301-48-2; [10] John Fox. An R and S-Plus Companion to Applied Regression. Sage Publications, Thousand Oaks, Calif., USA, 2002. ISBN 0761922792; [11] Peter Dalgaard. Introductory Statistics with R. Springer, 2002. ISBN 0-387-95475-9; [12] Stefano Iacus and Guido Masarotto. Laboratorio di statistica con R. McGraw-Hill, Milano, 2003. ISBN 88-386-6084-0; [13] John Maindonald and John Braun. Data Analysis and Graphics Using R. Cambridge University Press, Cambridge, 2003. ISBN 0-521-81336-0; [14] Giovanni Pannigiani, Elizabeth S. Garrett, Rafael A. Irizarry, and Scott L. Zeger. The Analysis of Gene Expression Data. Springer, New York, 2003. ISBN 0-387-95577-1; [15] Sylvie Huet, Annie Bouvier, Marie-Anne Gruet, and Emmanuel Jolivet. Statistical Tools for Nonlinear Regression. Springer, New York, 2003. ISBN 0-387-40081-8; [16] S. Mase, T. Kamakura, M. Jimbo, and K. Kanefuji. Introduction to Data Science for engineers—Data analysis using free statistical software R (in Japanese). Suuri-Kogaku-sha, Tokyo, April 2004. ISBN 4901683128; [17] Julian J. Faraway. Linear Models with R. Chapman & Hall/CRC, Boca Raton, Fla., 2004. ISBN 1-584-88425-8; [18] Richard M. Heiberger and Burt Holland. Statistical Analysis and Data Display: An Intermediate Course with Examples in S-Plus, R, and SAS. Springer Texts in Statistics. Springer, 2004. ISBN 0-387-40270-5; [19] John Verzani. Using R for Introductory Statistics. Chapman & Hall/CRC, Boca Raton, Fla., 2005. ISBN 1-584-88450-9; [20] Uwe Ligges. Programmieren mit R. Springer-Verlag, Heidelberg, 2005. ISBN 3-540-20727-9, in German; [21] Fionn Murtagh. Correspondence Analysis and Data Coding with JAVA and R. Chapman & Hall/CRC, Boca Raton, Fla., 2005. ISBN 1-584-88528-9; [22] Paul Murrell. R Graphics. Chapman & Hall/CRC, Boca Raton, Fla., 2005. ISBN 1-584-88486-X; [23] Michael J. Crawley. Statistics: An Introduction using R. Wiley, 2005. ISBN 0-470-02297-3; [24] Brian S. Everitt. An R and S-Plus Companion to Multivariate Analysis. Springer, 2005. ISBN 1-85233-882-2; [25] Richard C. Deonier, Simon Tavare, and Michael S. Waterman. Computational Genome Analysis: An Introduction. Springer, 2005. ISBN: 0-387-98785-1; [26] Robert Gentleman, Vince Carey, Wolfgang Huber, Rafael Irizarry, and Sandrine Dudoit, editors. Bioinformatics and Computational Biology Solutions Using R and Bioconductor. Statistics for Biology and Health. Springer, 2005. ISBN: 0-387-25146-4; [27] Terry M. Themeau and Patricia M. Grambsch. Modeling Survival Data: Extending the Cox Model. Statistics for Biology and Health. Springer, 2000. ISBN: 0-387-98784-3).

Unlike that of the Khvorova's paper, the dataset extracted from the Amarzguioui's paper divide the effective/ineffective groups on a basis of 70% inhibition efficiency of the expression. That is, the difference is expected to be shown more precisely in comparison with the success rate of prediction of the two scoring method in this data set. Table shows the results.

TABLE 8

| | Relative binding energy pattern | Dharmacon |
|---|---|---|
| LDA | 0.652 | 0.586 |
| QDA | 0.657 | 0.521 |

Referring to Table 8, the success rate of prediction is shown to be higher by 10% in the scoring method binding energy according to the present invention using the relative binding energy pattern than in the conventional scoring method of siRNA efficiency in both cases of LDA and QDA.

### Example 2

### Inhibition Experiment of Surviving Gene Expression

Through the siRNA design optimizing method according to the present invention using the relative binding energy pattern, 36 siRNAs for inhibiting surviving gene expression were designed, and then the inhibition experiment of the surviving gene expression was performed. The resultant data set was divided into effective/ineffective groups on a basis of 75% inhibition efficiency of expression. Here, the three data sets obtained from the Khvorova's paper and the Amarzguioui's paper were used as train sets, and the surviving data set was used as a test set. In the same way of Example 1, the score of siRNA was marked, and the success rate of prediction of the efficiency of siRNAs was calculated through LDA (Linear discriminant analysis) and QDA (Quadratic discriminant analysis) using the statistical program R. As a result, the success rate of prediction was 0.64 in both cases of LDA and QDA to show almost the same results of Example 1 (see Table 9).

TABLE 9

| | | | | | | |
|---|---|---|---|---|---|---|
| | Exp. ID | | SEQ ID | Knock | Z | Precise |
| NO | number | Sequence (3′ overhang: TT) | NO: | Down(%) | score | prediction |

| | | | | | | |
|---|---|---|---|---|---|---|
| 1 | 570(D) | GCAAUGUCUUAGGAAAGGA | 37 | >90 | 62.83 | 0 |
| 2 | 1106(D) | AGAAUAHCACAAACUACAA | 38 | >90 | 53.31 | 0 |
| 3 | 1189(D) | GAGACAGAAUAGAGUGAUA | 39 | >90 | 72.15 | 0 |
| 4 | 1212(Q) | GGGUCUGGCAGAUACUCCU | 40 | >90 | 68.48 | 0 |
| 5 | 299(AS) | UGCGCUUUGGUUUCUGUCA | 41 | 75-90 | 40.89 | |
| 6 | 319(G) | GAAGCAGUUUGAAGAAUUA | 42 | 75-90 | 64.37 | 0 |
| 7 | 574(Q)572 | UGUCUUAGGAAAGGAGAUC | 43 | 75-90 | 50.92 | 0 |
| 8 | 783(Q) | GGCAGUGUCCGUUUUGCUA | 44 | 75-90 | 57.52 | 0 |
| 9 | 1099(AS) | AAUUCACAGAAUAGCACAA | 45 | 75-90 | 46.80 | |
| 10 | 1133(D) | AAGCACAAAGCCAUUCUAA | 46 | 75-90 | 53.35 | 0 |
| 11 | 1305(Q) | GGCAGUGGCCUAAAUCCUU | 47 | 75-90 | 69.63 | 0 |
| 12 | 1480(G) | GGCUGAAGUCUGGCGUAAG | 48 | 75-90 | 50.20 | 0 |
| 13 | 1481(G) | GCUGAAGUCUGGCGUAAGA | 49 | 75-90 | 45.91 | |
| 14 | 1585(G) | CGGCUGUUCCUGAGAAAUA | 50 | 75-90 | 72.72 | 0 |
| 15 | 92(D) | AAGGACCACCGCAUCUCUA | 51 | 50-75 | 41.57 | 0 |
| 16 | 94(Q)92 | GGACCACCGCAUCUCUACA | 52 | 50-75 | 71.82 | |
| 17 | 294(G) | CGGGUUGCGCUUUCCUUUC | 53 | 50-75 | 44.18 | 0 |
| 18 | 693(D) | GCUGCUUCUCUCUCUCUCU | 54 | 50-75 | 63.54 | |
| 19 | 1021(G) | GUGAUGAGAGAAUGGAGAC | 55 | 50-75 | 57.86 | |
| 20 | 1188(G) | GGAGACAGAAUAGAGUGAU | 56 | 50-75 | 57.44 | |
| 21 | 1394(Q) | CCUUCACAUCUGUCACGUU | 57 | 50-75 | 57.48 | |
| 22 | 1546(G) | GAUUGUUACAGCUUCGCUG | 58 | 50-75 | 57.37 | |
| 23 | 90(AS) | UCAAGGACCACCGCAUCUC | 59 | <50 | 29.75 | 0 |
| 24 | 95(G) | GACCACCGCAUCUCUACAU | 60 | <50 | 55.86 | |
| 25 | 294(Q)282 | AAGCAUUCGUCCGGUUGCG | 61 | <50 | 18.86 | 0 |
| 26 | 289(D) | UUCGUCCGGUUGCGCUUUG | 62 | <50 | 39.01 | 0 |
| 27 | 428(Q)426 | ACUGCGAAGAAAGUGCGCC | 63 | <50 | 23.96 | 0 |
| 28 | 780(Q)778 | GAAGGCAGUGUCCCUUUUG | 64 | <50 | 56.04 | |
| 29 | 807(G) | GACAGCUUUGUUCGCGUGG | 65 | <50 | 43.89 | 0 |
| 30 | 846(Q) | UGUGUCUGGACCUCAUGUU | 66 | <50 | 47.41 | 0 |
| 31 | 1130(Q) | ACUAAGCACAAAGCCAUUC | 67 | <50 | 47.75 | 0 |
| 32 | 1141(Q) | AGCCAUUCUAAGUCAUUGG | 68 | <50 | 33.49 | 0 |
| 33 | 1142(Q) | GCCAUUGUAAGUCAUUGGG | 69 | <50 | 37.58 | 0 |
| 34 | 1236(D) | CACUGCUGUGUGAUUAGAC | 70 | <50 | 35.92 | 0 |
| 35 | 1325(D) | UUAAAUGACUUGGCUCGAU | 71 | <50 | 52.86 | |
| 36 | 1390(G) | CCAACCUUCACAUCUGUCA | 72 | <50 | 63.50 | |
| | | Total success rate | | | | 23 |
| | | (23/36) = 64% | | | | |

### INDUSTRIAL APPLICABILITY

As described above, according to the method of the present invention, a researcher or an experimenter can analyzes patterns of a relative binding energy on base sequences of unknown siRNA without actual experiments to determine whether the siRNA is effective or ineffective rapidly, thereby design and production efficiency of siRNA can be maximized and target mRNA expression can be effectively inhibited with efficient siRNA to the target mRNA.

### Sequence List

Attached

1. A method of inhibiting target mRNA expression using siRNA, comprising the steps of:
(1) obtaining all combinations of ds (double strand) RNA sequences each of which consists of n numbers of nucleotides complementary to a predetermined target mRNA (n is an integer);
(2) obtaining E_{A}, E_{B}, E_{C} and E_{D} with respect to each dsRNA, which are mean binding energy values of 1^{st}-2^{nd} section (A), 3^{rd}-7^{th} section (B), 8^{th}-15^{th} section (C) and 16^{th}-18^{th} section (D) in the base sequence of the dsRNA, respectively;
(3) allotting Y_{(A-B)}, Y_{(B-C)}, Y_{(C-D)} and Y_{(A-D)} to each section of (A) through (D) according to the following equation,
i) in case of −0.02<E_{A}−E_{B}<0.38, −0.29<E_{B}−E_{C}<−0.01, 0.00<E_{C}−E_{D}<0.35, 0.07<E_{D}−E_{A}<0.37, then each of Y_{(A-B)}, Y_{(B-C)}, Y_{(C-D)} and Y_{(A-D)} is 10 point,
ii) in case of −0.63<E_{A}−E_{B}<−0.21, 0.05<E_{B}−E_{C}<0.44, −0.47<E_{C}−E_{D}<−0.09, −0.67<E_{D}−E_{A}<−0.23, each of Y_{(A-B)}, Y_{(B-C)}, Y_{(C-D)} and Y_{(A-D)} is 0 point,
iii) in case of E_{A}−E_{B}, E_{B}−E_{C}, E_{C}−E_{D} and E_{D}−E_{A} being out of range defined in (i) and (ii), each of Y_{(A-B)}, Y_{(B-C)}, Y_{(C-D)} and Y_{(A-D)} is 5 point;
(4) allotting a relative binding energy value Y value with respect to each dsRNA according to the following Equation 4:
$$Y = \frac{\begin{matrix}{W}_{( A - B )} {Y}_{( A - B )} + {W}_{( B - C )} {Y}_{( B - C )} + \\ {W}_{( C - D )} {Y}_{( C - D )} + {W}_{( A - D )} {Y}_{( A - D )}\end{matrix}}{10 ( {W}_{( A - B )} + {W}_{( B - C )} + {W}_{( C - D )} + {W}_{( A - D )} )} \times 100 \qquad [ Equation 4 ]$$
wherein W_{(A-B)}, W_{(B-C)}, W_{(C-D)} and W_{(A-D)} are weights for sections (A-B), (B-C), (C-D) and (A-D) which ranges from 0.90 to 1.00, 0.2 to 0.4, 0.2 to 0.3 and 0.7 to 0.9, respectively;
(5) allotting Z value with respect to each dsRNA according to the following Equation 5:
$$Z = 100 \times \frac{{\sum }_{i} {W}_{i} \frac{{Z}_{i}}{{M}_{i}}}{{\sum }_{i} {W}_{i}} \qquad [ Equation 5 ]$$
wherein i is an integer representing a factor affecting siRNA's inhibition efficiency on the target mRNA, at least one of which is the relative binding energy of the siRNA,
Zᵢ is a point given to each factor, provided that Z₁=Y, representing a relative binding energy,
Mᵢ is a predetermined maximum value allotted to each factor, and
Wᵢ is a predetermined weight allotted to each factor based on W₁;
(6) arranging Z values obtained from the step (5) in a descending order with respect to each dsRNA to select predetermined top % of dsRNAs; and
(7) applying the selected dsRNAs to inhibit the target mRNA expression.

2. The method according to claim 1, wherein the siRNA is double strand RNA of 21 nucleotides where n is 21.

3. The method according to claim 1, wherein the siRNA has an overhang structure of 1 to 3 nucleotide at the dsRNA portion and both side 3′-ends of 19 nucleotides.

4. The method according to claim 1, wherein the weighting factors W_{(A-B)}, W_{(B-C)}, W_{(C-D)} and W_{(A-D)} are individually 1.00, 0.37, 0.20 and 0.90.

5. The method according to claim 1, wherein the factor that affects inhibition efficiency of siRNA to target mRNA in the step (5) includes a relative binding energy as an essential factor, and one or more factors selected from the group comprising the number of A/U in 5 bases of 3′-end, the presence of G/C at 1^{st} position, the presence of A/U at 19^{th} position, the content of G/C, Tₘ, secondary structure of RNA, homology with other mRNA as an optional factor.

6. The method according to claim 1, wherein the Equation 5 of the step (5) is characterized in that I=5; Z₁=relative binding energy point (Y), Z₂=point allotted to the number of A/U in 5 bases of 3′-end, Z₃=point allotted to the presence of G/C at 1^{st} position, Z₄=point allotted to the presence of A/U at 19^{th} position, and Z₅=point allotted to the content of G/C; M₁-M₅ are individually 100, 5, 1, 1, 10; W₁-W₅ are individually 0.90, 0.07, 0.15, 0.19, 0.11.

7. The method according to claim 1, wherein the predetermined % of the step (5) is upper 10%.

8. A method of inhibiting target mRNA expression using siRNA, comprising the steps of:
(1) obtaining all combination of ds (double strand) RNA sequences each of which consists of n numbers of nucleotides complementary to a predetermined target mRNA (n is an integer);
(2) obtaining E_{A}, E_{B}, E_{C} and E_{D} with respect to each dsRNA, which are mean binding energy values of 1^{st}-2^{nd} section (A), 3^{rd}-6^{th} section (B), 14^{th}-16^{th} section (C) and 16^{th}-18^{th} section (D) in the base sequence of the dsRNA, respectively;
(3) allotting Y_{(A-B)}, Y_{(B-C)}, Y_{(C-D)} and Y_{(A-D)} to each section of (A) through (D) according to the following equation
i) in case of 0.00<E_{A}−E_{B}<0.40, −0.41<E_{B}−E_{C}<−0.01, 0.07<E_{C}−E_{D}<0.39, 0.07<E_{D}−E_{A}<0.37, then each of Y_{(A-B)}, Y_{(B-C)}, Y_{(C-D)} and Y_{(A-D)} is 10 point,
ii) in case of −0.63<E_{A}−E_{B}<−0.21, 0.10<E_{B}−E_{C}<0.51, −0.47<E_{C}−E_{D}<−0.19, −0.67<E_{D}−E_{A}<−0.23, each of Y_{(A-B)}, Y_{(B-C)}, Y_{(C-D)} and Y_{(A-D)} is 0 point,
iii) in case of E_{A}−E_{B}, E_{B}−E_{C}, E_{C}−E_{D} and E_{D}−E_{A} being out of range defined in (i) and (ii), each of Y_{(A-B)}, Y_{(B-C)}, Y_{(C-D)} and Y_{(A-D)} is 5 point;
(4) allotting a relative binding energy Y value with respect to each dsRNA according to the following Equation 4:
$$Y = \frac{\begin{matrix}{W}_{( A - B )} {Y}_{( A - B )} + {W}_{( B - C )} {Y}_{( B - C )} + \\ {W}_{( C - D )} {Y}_{( C - D )} + {W}_{( A - D )} {Y}_{( A - D )}\end{matrix}}{10 ( {W}_{( A - B )} + {W}_{( B - C )} + {W}_{( C - D )} + {W}_{( A - D )} )} \times 100 \qquad [ Equation 4 ]$$
wherein W_{(A-B)}, W_{(B-C)}, W_{(C-D)} and W_{(A-D)} are individually weights for sections (A-B), (B-C), (C-D) and (A-D) which ranges from 0.5 to 0.7, 0.3 to 0.5, 0.3 to 0.5 and 0.9 to 1.0, respectively;
(5) allotting Z value with respect to each dsRNA according to the following Equation 5:
$$Z = 100 \times \frac{{\sum }_{i} {W}_{i} \frac{{Z}_{i}}{{M}_{i}}}{{\sum }_{i} {W}_{i}} \qquad [ Equation 5 ]$$
wherein i is an integer representing a factor affecting siRNA's inhibition efficiency on the target mRNA, at least one of which is the relative binding energy of siRNA,
Z₁ is a point given to each factor, provided that Zᵢ=Y, representing a relative binding energy point,
Mᵢ is a predetermined maximum value allotted to each factor, and
Wᵢ is a predetermined weight allotted to each factor based on W₁;
(6) arranging Z values obtained from the step (5) in a descending order with respect to each dsRNA to select predetermined top % of dsRNAs; and
(7) applying the selected dsRNAs to inhibit the target mRNA expression.

9. The method according to claim 8, wherein the siRNA is double strand RNA of 21 nucleotides where n is 21.

10. The method according to claim 8 or 9, wherein the siRNA has an overhang structure of 1 to 3 nucleotide at the dsRNA portion and both side 3′-ends of 19 nucleotides.

11. The method according to claim 8, wherein the weighting factors W_{(A-B)}, W_{(B-C)}, W_{(C-D)} and W_{(A-D)} are individually 0.65, 0.48, 0.48 and 0.90.

12. The method according to claim 8, wherein the factor that affects inhibition efficiency of siRNA to target mRNA in the step (5) includes a relative binding energy as an essential factor, and one or more factors selected from the group comprising the number of A/U in 5 bases of 3′-end, the presence of G/C at 1^{st} position, the presence of A/U at 19^{th} position, the content of G/C, Tₘ, secondary structure of RNA, homology with other mRNA as an optional factor.

13. The method according to claim 8, wherein the Equation 5 of the step (5) is characterized in that i=5; Z₁=relative binding energy point (Y), Z₂=point allotted to the number of A/U in 5 bases of 3′-end, Z₃=point allotted to the presence of G/C at 1^{st} position, Z₄=point allotted to the presence of A/U at 19^{th} position, and Z₅=point allotted to the content of G/C; M₁-M₅ are individually 100, 5, 1, 1, 10; and W₁-W₅ are individually 0.90, 0.07, 0.15, 0.19, 0.11.

14. The method according to claim 8, wherein the predetermined % of the step (5) is upper 10%.

15. A method of optimizing siRNA design, comprising the steps of;
(1) obtaining all combinations of ds (double strand) RNA sequences each of which consists of n numbers of nucleotides complementary to a predetermined target mRNA (n is an integer);
(2) obtaining E_{A}, E_{B}, E_{C} and E_{D} with respect to each dsRNA, which are mean binding energy values of 1^{st}-2^{nd} section (A), 3^{rd}-7^{th} section (B), 8^{th}-15^{th} section (C) and 16^{th}-18^{th} section (D) in the base sequence of the dsRNA, respectively;
(3) allotting Y_{(A-B)}, Y_{(B-C)}, Y_{(C-D)} and Y_{(A-D)} to each section of (A) through (D) according to the following equation,
i) in case of −0.02<E_{A}-E_{B}<0.38, −0.29<E_{B}−E_{C}<−0.01, 0.00<E_{C}−E_{D}<0.35, 0.07<E_{D}−E_{A}<0.37, then each of Y_{(A-B)}, Y_{(B-C)}, Y_{(C-D)} and Y_{(A-D)} is 10 point,
ii) in case of −0.63<E_{A}−E_{B}<−0.21, 0.05<E_{B}−E_{C}<0.44, −0.47<E_{C}−E_{D}<−0.09, −0.67<E_{D}−E_{A}<−0.23, each of Y_{(A-B)}, Y_{(B-C)}, Y_{(C-D)} and Y_{(A-D)} is 0 point,
iii) in case of E_{A}−E_{B}, E_{B}−E_{C}, E_{C}−E_{D} and E_{D}−E_{A} being out of range defined in (i) and (ii), each of Y_{(A-B)}, Y_{(B-C)}, Y_{(C-D)} and Y_{(A-D)} is 5 point;
(4) allotting a relative binding energy value Y value with respect to each dsRNA according to the following Equation 4:
$$Y = \frac{\begin{matrix}{W}_{( A - B )} {Y}_{( A - B )} + {W}_{( B - C )} {Y}_{( B - C )} + \\ {W}_{( C - D )} {Y}_{( C - D )} + {W}_{( A - D )} {Y}_{( A - D )}\end{matrix}}{10 ( {W}_{( A - B )} + {W}_{( B - C )} + {W}_{( C - D )} + {W}_{( A - D )} )} \times 100 \qquad [ Equation 4 ]$$
wherein W_{(A-B)}, W_{(B-C)}, W_{(C-D)} and W_{(A-D)} are weights for sections (A-B), (B-C), (C-D) and (A-D) which ranges from 0.90 to 1.00, 0.2 to 0.4, 0.2 to 0.3 and 0.7 to 0.9, respectively;
(5) allotting Z value with respect to each dsRNA according to the following Equation 5:
$$Z = 100 \times \frac{{\sum }_{i} {W}_{i} \frac{{Z}_{i}}{{M}_{i}}}{{\sum }_{i} {W}_{i}} \qquad [ Equation 5 ]$$
wherein i is an integer representing a factor affecting siRNA's inhibition efficiency on the target mRNA, at least one of which is the relative binding energy of the siRNA,
Zᵢ is a point given to each factor, provided that Z₁=Y, representing a relative binding energy,
Mᵢ is a predetermined maximum value allotted to each factor, and
Wᵢ is a predetermined weight allotted to each factor based on W₁; and
(6) arranging Z values obtained from the step (5) in a descending order with respect to each dsRNA to select predetermined top % of dsRNAs.

16. A method of optimizing siRNA design, comprising the steps of:
(1) obtaining all combination of ds (double strand) RNA sequences each of which consists of n numbers of nucleotides complementary to a predetermined target mRNA (n is an integer);
(2) obtaining E_{A}, E_{B}, E_{C} and E_{D} with respect to each dsRNA, which are mean binding energy values of 1^{st}-2^{nd} section (A), 3^{rd}-6^{th} section (B), 14^{th}-16^{th} section (C) and 16^{th}-18^{th} section (D) in the base sequence of the dsRNA, respectively;
(3) allotting Y_{(A-B)}, Y_{(B-C)}, Y_{(C-D)} and Y_{(A-D)} to each section of (A) through (D) according to the following equation
i) in case of 0.00<E_{A}−E_{B}<0.40, −0.41<E_{B}−E_{C}<−0.01, 0.07<E_{C}−E_{D}<0.39, 0.07<E_{D}−E_{A}<0.37, then each of Y_{(A-B)}, Y_{(B-C)}, Y_{(C-D)} and Y_{(A-D)} is 10 point,
ii) in case of −0.63<E_{A}−E_{B}<−0.21, 0.10<E_{B}−E_{C}<0.51, −0.47<E_{C}−E_{D}<−0.19, −0.67<E_{D}−E_{A}<−0.23, each of Y_{(A-B)}, Y_{(B-C)}, Y_{(C-D)} and Y_{(A-D)} is 0 point,
iii) in case of E_{A}−E_{B}, E_{B}−E_{C}, E_{C}−E_{D} and E_{D}−E_{A} being out of range defined in (i) and (ii), each of Y_{(A-B)}, Y_{(B-C)}, Y_{(C-D)} and Y_{(A-D)} is 5 point;
(4) allotting a relative binding energy Y value with respect to each dsRNA according to the following Equation 4:
$$Y = \frac{\begin{matrix}{W}_{( A - B )} {Y}_{( A - B )} + {W}_{( B - C )} {Y}_{( B - C )} + \\ {W}_{( C - D )} {Y}_{( C - D )} + {W}_{( A - D )} {Y}_{( A - D )}\end{matrix}}{10 ( {W}_{( A - B )} + {W}_{( B - C )} + {W}_{( C - D )} + {W}_{( A - D )} )} \times 100 \qquad [ Equation 4 ]$$
wherein W_{(A-B)}, W_{(B-C)}, W_{(C-D)} and W_{(A-D)} are individually weights for sections (A-B), (B-C), (C-D) and (A-D) which ranges from 0.5 to 0.7, 0.3 to 0.5, 0.3 to 0.5 and 0.9 to 1.0, respectively;
(5) allotting Z value with respect to each dsRNA according to the following Equation 5:
$$Z = 100 \times \frac{{\sum }_{i} {W}_{i} \frac{{Z}_{i}}{{M}_{i}}}{{\sum }_{i} {W}_{i}} \qquad [ Equation 5 ]$$
wherein i is an integer representing a factor affecting siRNA's inhibition efficiency on the target mRNA, at least one of which is the relative binding energy of siRNA,
Z₁ is a point given to each factor, provided that Z₁=Y, representing a relative binding energy point,
Mᵢ is a predetermined maximum value allotted to each factor, and
Wᵢ is a predetermined weight allotted to each factor based on W₁; and
(6) arranging Z values obtained from the step (5) in a descending order with respect to each dsRNA to select predetermined top % of dsRNAs.

17. The method according to claim 2, wherein the siRNA has an overhang structure of 1 to 3 nucleotide at the dsRNA portion and both side 3′-ends of 19 nucleotides.

18. The method according to claim 5, wherein the Equation 5 of the step (5) is characterized in that i=5; Z₁=relative binding energy point (Y), Z₂=point allotted to the number of A/U in 5 bases of 3′-end, Z₃=point allotted to the presence of G/C at 1^{st} position, Z₄=point allotted to the presence of A/U at 19^{th} position, and Z₅=point allotted to the content of G/C; M₁-M₅ are individually 100, 5, 1, 1, 10; W₁-W₅ are individually 0.90, 0.07, 0.15, 0.19, 0.11.

19. The method according to claim 9, wherein the siRNA has an overhang structure of 1 to 3 nucleotide at the dsRNA portion and both side 3′-ends of 19 nucleotides.

20. The method according to claim 12, wherein the Equation 5 of the step (5) is characterized in that i=5; Z₁=relative binding energy point (Y), Z₂=point allotted to the number of A/U in 5 bases of 3′-end, Z₃=point allotted to the presence of G/C at 1^{st} position, Z₄=point allotted to the presence of A/U at 19^{th} position, and Z₅=point allotted to the content of G/C; M₁-M₅ are individually 100, 5, 1, 1, 10; and W₁-W₅ are individually 0.90, 0.07, 0.15, 0.19, 0.11.
